(19) United States
(12) Patent Application Publication
(10) Pub. No.: US 2026/0221245 A1
(43) Pub. Date: Jul. 30, 2026

(54) COMPUTERIZED SYSTEM TO PROVIDE MEDICAL DIAGNOSIS, PROGNOSIS, AND TREATMENT USING MORE REFINED DIGITAL HEALTH RECORDS HAVING IMPROVED CONTEXT

(71) Applicant: MD Aware LLC, New York, NY (US)

(72) Inventors: Joseph Habboushe, New York, NY (US); Graham Walker, San Francisco, CA (US)

(21) Appl. No.: 19/578,776

(22) Filed: Mar. 25, 2026

Related U.S. Application Data
(63) Continuation-in-part of application No. 18/787,719, filed on Jul. 29, 2024.
Application No. 18/787,719 is a continuation-in-part of application No. 17/102,328, filed on Nov. 23, 2020, now abandoned.
Application No. 17/102,328 is a continuation-in-part of application No. 15/416,831, filed on Jan. 26, 2017, now abandoned.
(60) Provisional application No. 62/289,831, filed on Feb. 1, 2016.

(51) Int. Cl. G16H 10/60; G06F 21/62; G16H 50/20

(52) U.S. Cl. CPC G16H 10/60; G06F 21/6245; G16H 50/20

(57) ABSTRACT

A computer-implemented system improves clinical decision support by combining deterministic tool execution with anonymized machine-learning inference and gated write-back to an electronic health record (EHR). The system accesses an EHR, selects a decision-support tool with an input schema, and forms a bounded patient-data set of EHR items relevant to required input fields. Structured input values are derived and executed by a deterministic calculator engine to produce a tool output. An inference request derived from the bounded patient-data set is routed through an anonymization proxy that removes or replaces patient-identifying tokens and withholds a session identifier before transmitting to a remote inference computing system hosting a trained machine learning model, which returns a narrative output. A verification engine detects disagreement between the narrative and tool outputs and validates one or more constraints. A policy enforcement gateway selectively permits or blocks write-back.

### CROSS REFERENCE TO RELATED APPLICATIONS

This application is a continuation-in-part under 35 U.S.C. § 120 of U.S. patent application Ser. No. 18/787,719, filed Jul. 29, 2024, which is a continuation-in-part under 35 U.S.C. § 120 of U.S. patent application Ser. No. 17/102,328, filed Nov. 23, 2020, which is a continuation-in-part under 35 U.S.C. § 120 of U.S. patent application Ser. No. 15/416,831, filed Jan. 26, 2017, which claims the benefit under 35 U.S.C. § 119(e) of U.S. Provisional Patent Application No. 62/289,831, filed Feb. 1, 2016, the disclosures of which are hereby incorporated by reference, in their entireties, for all purposes.

### BACKGROUND

Digital records of patient health, such as electronic health records (EHRs) and electronic medical records (EMRs), are recorded by medical professionals to document patient data which may include diagnoses, medications, immunizations, and family medical histories. In their busy schedules, medical professionals have limited time to update the digital records. To expedite the entry of the digital records, medical professionals may utilize abbreviations, acronyms, or other shortcuts to save time. However, such entries may be ambiguous or incomplete to other viewers of the digital records, such as other medical professionals or patients. These ambiguous or incomplete entries may be catalysts that could lead to downstream consequences such as incorrect prescriptions or improper diagnosis, prognosis, or treatment. Incorrect prescriptions in the United States alone result in death of an estimated 7,000 to 9,000 people per year. Thus, the necessity of accurate, clear, and complete digital records cannot be overstated.

### SUMMARY

A system of one or more computers can be configured to perform particular operations or actions by virtue of having software, firmware, hardware, or any combination thereof installed on the system that, in operation, causes the system to perform the actions. One or more computer programs can be configured to perform particular operations or actions by virtue of including instructions that, when executed by data-processing apparatus, cause the apparatus to perform the actions.

In one general aspect, a computer-implemented system includes accessing an electronic health record (EHR) of a patient from a digital health data platform. The computer-implemented system further includes selecting a clinical decision-support (CDS) tool from a repository of CDS tools, where the selected CDS tool has an input schema defining required input fields. The system further includes constructing, for the selected CDS tool, a bounded patient-data set by selecting, from the EHR, patient data items relevant to the input schema, and determining, from the bounded patient-data set, structured input values for the required input fields of the input schema. The system further includes executing the selected CDS tool using a deterministic engine, implemented as at least one of an in-process software module or a network-accessible calculator service, to generate a tool output based on the structured input values.

The system further includes routing, via a network-accessible anonymization proxy, an inference request derived from at least a portion of the bounded patient-data set to a remote inference computing system that hosts a trained machine learning model. The anonymization proxy generates an anonymized inference request by removing or replacing patient-identifying tokens and withholding at least one session identifier before transmitting the anonymized inference request to the remote inference computing system, and the system receives a narrative output from the remote inference computing system. The system further includes performing, by a verification engine, a hybrid verification that includes (i) detecting disagreement between the narrative output and the tool output and (ii) verifying one or more validation constraints for at least one of the structured input values. Based on results of the hybrid verification, the system selectively permits or blocks, via a policy enforcement gateway, write-back of at least one of the tool output or a revision to the EHR to the digital health data platform, and stores an audit record linking a write-back decision to the bounded patient-data set and the results of the hybrid verification. Other embodiments of this aspect include corresponding computer systems, apparatuses, and computer programs recorded on one or more computer storage devices, each configured to perform the actions described herein.

Implementations may include one or more of the following features. In some implementations, selecting the CDS tool includes obtaining ambient note-taking content associated with a clinical encounter; converting the ambient note-taking content into textual content using one or more speech or text processing operations; extracting one or more clinical concepts from the textual content; generating a workflow-context representation based on the one or more clinical concepts and at least one portion of the EHR; and selecting the CDS tool from the repository based on a correspondence between the workflow-context representation and tool metadata for the CDS tool. In some implementations, determining the structured input values includes mapping at least one patient data item to a required input field using a tool-specific parameter mapping function stored in association with the selected CDS tool in the repository, where, in response to multiple candidate patient data items corresponding to a same required input field, the determining includes resolving a conflict among the multiple candidate patient data items according to a resolution policy. In some implementations, the resolution policy prioritizes sensor-verified measurements over manually entered values, more recent measurements over older measurements, or measurements corroborated by multiple EHR sections over measurements appearing in a single EHR section.

In some implementations, the verification engine verifies the one or more validation constraints by applying a tool-specific constraint defined in the repository, where the tool-specific constraint includes an allowed numeric range, an allowed unit set, a required recency threshold, and a required completeness threshold for the required input fields. In some implementations, detecting disagreement between the narrative output and the tool output includes determining that the narrative output asserts a numeric score, category, threshold crossing, or recommended action that is inconsistent with a corresponding value in the tool output by more than a disagreement threshold. In some implementations, responsive to detecting the disagreement, the policy enforcement gateway prevents write-back of the narrative output and authorizes write-back of a representation of the tool output that omits the inconsistent portion of the narrative output. In some implementations, the anonymization proxy withholds the at least one session identifier by terminating a first secure communication session with a client device and originating a second secure communication session with the remote inference computing system, such that transport-layer identifiers of the first secure communication session are not forwarded to the remote inference computing system. In some implementations, removing or replacing patient-identifying tokens includes replacing at least one patient identifier with a placeholder token, storing a placeholder-to-identifier mapping locally to the digital health data platform, and preventing transmission of the placeholder-to-identifier mapping to the remote inference computing system.

In some implementations, the audit record includes an identifier of the selected CDS tool, identifiers of patient data items in the bounded patient-data set used to determine the structured input values, the tool output, an indication of whether disagreement was detected, and a write-back decision outcome. In some implementations, responsive to determining that a required input field is missing from the structured input values, the verification engine generates a request for user confirmation or entry for the missing required input field, and the deterministic engine executes the selected CDS tool after receiving the user confirmation or entry. In some implementations, the system stores, for the selected CDS tool, a cached tool-execution artifact that includes the structured input values and the tool output, and invalidates the cached tool-execution artifact responsive to detecting a change to an EHR data item that was used to determine at least one of the structured input values. In some implementations, selecting the CDS tool further includes ranking a plurality of candidate CDS tools based on at least one of historical usage frequency for similar workflow contexts, historical user acceptance of tool outputs for similar workflow contexts, or similarity between the bounded patient-data set and prior bounded patient-data sets associated with prior tool executions, and selecting a top-ranked CDS tool. In some implementations, determining the structured input values includes applying a trained machine learning model to map unstructured encounter text to required input fields of the input schema, where the trained machine learning model is trained using training examples that pair (i) historical encounter text and (ii) corresponding confirmed input-field values used to execute the CDS tool. Implementations of the described techniques may include hardware, a method or process, or a computer tangible medium.

In one general aspect, a computer-implemented method includes accessing an electronic health record (EHR) of a patient from a digital health data platform. The method further includes selecting a clinical decision-support (CDS) tool from a repository of CDS tools, where the selected CDS tool is associated with an input definition identifying one or more input fields. The method further includes determining, for the selected CDS tool, a patient-data set by selecting from the EHR patient data items relevant to the one or more input fields; determining, based on the patient-data set, one or more input values corresponding to the one or more input fields; and executing the selected CDS tool using the one or more input values to generate a tool output. The method further includes transmitting, via an anonymization component, a request derived from at least a portion of the patient-data set to a remote computing system that generates a narrative output, where the anonymization component generates an anonymized request by removing or replacing at least one patient identifier and withholding at least one session-related identifier before transmitting the anonymized request. The method further includes performing a verification that includes (i) evaluating consistency between the narrative output and the tool output and (ii) evaluating at least one validation condition for at least one of the one or more input values. Based on the verification, the method controls whether to update the digital health data platform with at least one of the tool output or a revision to the EHR, and stores an audit record associated with the update control. Other embodiments of this aspect include corresponding computer systems, apparatuses, and computer programs recorded on one or more computer storage devices, each configured to perform the actions described herein.

Implementations may include one or more of the following features. In some implementations, selecting the CDS tool includes obtaining ambient note-taking content associated with a clinical encounter; converting the ambient note-taking content into textual content using one or more speech or text processing operations; extracting one or more clinical concepts from the textual content; generating a workflow-context representation based on the one or more clinical concepts and at least one portion of the EHR; and selecting the CDS tool from the repository based on a correspondence between the workflow-context representation and tool metadata for the CDS tool. In some implementations, the anonymization component withholds the at least one session-related identifier by terminating a first secure communication session with a client device and originating a second secure communication session with the remote computing system such that a transport-layer identifier of the first secure communication session is not forwarded to the remote computing system. In some implementations, the method further includes storing, for the selected CDS tool, a cached tool-execution artifact that includes at least the one or more input values and the tool output, and invalidating the cached tool-execution artifact responsive to detecting a change to an EHR data item that was used to determine at least one of the one or more input values. Implementations of the described techniques may include hardware, a method or process, or a computer tangible medium.

In one general aspect, a non-transitory computer-readable medium stores instructions that, when executed, cause one or more processors to perform operations including accessing an electronic health record (EHR) of a patient from a digital health data platform; selecting a clinical decision-support (CDS) tool from a repository of CDS tools, where the selected CDS tool is associated with an input definition identifying one or more input fields; determining, for the selected CDS tool, a patient-data set by selecting from the EHR patient data items relevant to the one or more input fields; determining, based on the patient-data set, one or more input values corresponding to the one or more input fields; executing the selected CDS tool using the one or more input values to generate a tool output; transmitting, via an anonymization component, a request derived from at least a portion of the patient-data set to a remote computing system that generates a narrative output, where the anonymization component generates an anonymized request by removing or replacing at least one patient identifier and withholding at least one session-related identifier before transmitting the anonymized request; performing a verification that includes (i) evaluating consistency between the narrative output and the tool output and (ii) evaluating at least one validation condition for at least one of the one or more input values; and, based on the verification, controlling whether to update the digital health data platform with at least one of the tool output or a revision to the EHR, and storing an audit record associated with the update control. Other embodiments of this aspect include corresponding computer systems, apparatuses, and computer programs recorded on one or more computer storage devices, each configured to perform the actions described herein.

Implementations may include one or more of the following features. In some implementations, the anonymization component withholds the at least one session-related identifier by terminating a first secure communication session with a client device and originating a second secure communication session with the remote computing system such that a transport-layer identifier of the first secure communication session is not forwarded to the remote computing system. Implementations of the described techniques may include hardware, a method or process, or a computer tangible medium.

### BRIEF DESCRIPTION OF THE DRAWINGS

Certain features of various embodiments of the present technology are set forth with particularity in the appended claims. A better understanding of the features and advantages of the technology will be obtained by reference to the following detailed description that sets forth illustrative embodiments, in which the principles of the invention are utilized, and the accompanying drawings of which:

FIG. 1 illustrates an exemplary flow diagram of an improved medical diagnostic and treatment system, in accordance with an embodiment.

FIGS. 2A-2B illustrate exemplary implementations of a digital health data platform, in accordance with an embodiment.

FIGS. 3-4, 5A-5B, and 6-8 illustrate exemplary implementations of mechanisms of updating the digital health data platform, in accordance with an embodiment.

FIG. 9 illustrates a block diagram showing an integrated clinical computing system coupled to a digital health data platform storing an electronic health record (EHR), and further coupled to a remote inference computing system, in accordance with some embodiments.

FIG. 10 illustrates a diagram showing context-driven CDS tool selection and patient-data-to-input derivation, in accordance with some embodiments.

FIG. 11 illustrates anonymized inference routing and hybrid verification with policy-gated write-back, in accordance with some embodiments.

FIG. 12-13 illustrate exemplary implementation of diagnostic tests, methods, or protocols, prognostic methods, algorithms, and/or determinations of treatment options, which may be implemented in conjunction with any of the implementations of the previous FIGS. 1-8.

FIG. 14 illustrates a block diagram of a client-side multi-pseudonymization architecture for generating, transmitting, and evaluating parallel clinical inquiries to preserve privacy, in accordance with some embodiments.

FIG. 15 illustrates a flow diagram of a controlled perturbation and bias auditing workflow for detecting algorithmic bias through clinical disagreement, in accordance with some embodiments.

FIG. 16 illustrates a block/flow diagram of a cross-device session-pairing workflow in which a first client device displays a clinical response containing substitute tokens and, via a temporary proximity-based link to a second client device holding an exclusive local mapping, temporarily replaces the substitute tokens with corresponding PHI strings for a duration of a secure session, in accordance with some embodiments.

### DETAILED DESCRIPTION

Embodiments described in this application provide a method, implemented by a system having one or more computer processors, that enhances an accuracy, reliability, and completeness of digital health records of patients, while seamlessly completing and/or transmitting electronic prescriptions, and synchronizing with diagnostic and surgical equipment. The system may detect entries in the digital health records that have potentially ambiguous, inaccurate, or unreliable data, and provide suggestions such as clinical concepts to rectify such data. The system may further present alternative suggestions, receive an input of which, if any, suggestion has been accepted or adopted, and incorporate whichever suggestion has been accepted or adopted into the digital health records. The system may include a machine learning model that is trained over time to improve the suggestions so that the suggestions include more accurate, relevant or appropriate data and/or more closely account for conventions of a particular medical practice. Additionally, once the system receives updated data, the system may automatically and dynamically update other relevant entries of the digital health data platform so that a practitioner does not need to manually search for each entry that needs to be updated. In summary, the system provides options or suggestions to revise incorrect or ambiguous data that would otherwise remain unchecked and automatically update entries in digital health records that may otherwise be forgotten, while coordinating and initiating diagnostic, prognostic, and treatment options.

In some embodiments, the system may be configured to determine a diagnosis, prognosis, or treatment of a patient actually or potentially infected by COVID-19 and/or indicate a severity of risk of such a patient. The system may determine one or more tests, protocols, or methods is/are most appropriate based on data of the patient provided in the digital health data platform.

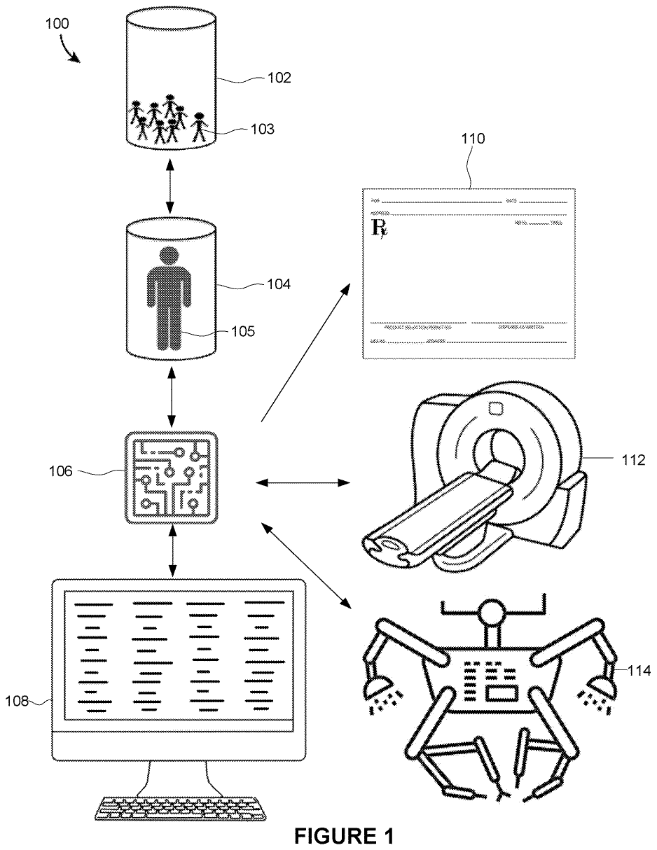

FIG. 1 illustrates an exemplary flow diagram of an improved medical diagnostic and treatment system. In FIG. 1, a database 102 may include medical data of patients 103. From the database 102, records 104 of a patient 105 may be selected, for example, on a device 108, by a user operating the device 108. The patient 105 may be a patient that is currently being operated on and/or a patient of interest, for example, of which further data is being gathered. One or more processors 106 may process and/or analyze the records 104, determine one or more suggestions to improve an accuracy, clarity, and/or completeness of the records 104 along with probabilities of relevance of each of the suggestions. In some embodiments, the suggestions may include terms as an addition, deletion, or replacement, based on a context of the records 104 of the patient 105, and/or of the patients 103 as a whole in the database 102. The one or more processors 106 may present, onto the device 108, suggestions and the probabilities of relevance. The one or more processors 106 may present the suggestions and the probabilities of relevance simultaneous with text being inputted, for example, on the device 108, or after text has been completely inputted. For example, text may be inputted by a user of the device 108. The device 108 may, in some embodiments, be a personal computer, a handheld device such as a mobile phone, tablet or any other device. The device 108 may receive an input regarding which, if any, suggestions have been adopted or accepted. The device 108 may transmit the input back to the one or more processors 106. The one or more processors 106 may update the records 104 of the patient 105 from the received input, thereby updating the database 102.

The one or more processors 106 may be trained to improve the suggestions and determination of probabilities of relevance based on which of the suggestions have been adopted or accepted. For example, if the input to the device 108 indicates that the suggestions weigh certain portions of the patient data, such as the problems list or the testing results, more heavily, the one or more processors 106 may be trained to determine and/or output suggestions that more heavily weigh those portions of the patient data. As another example, if the input to the device 108 indicates that the suggestions tend to have more definitive language, such as “concussion” as opposed to “possible concussion” or “probable concussion,” the one or more processors 106 may be trained to determine and/or output suggestions that include more definitive language. In some embodiments, multiple training sets may be utilized to train the one or more processors, for example, a first training set may include instances in which one of the alternative suggestions has been accepted, and a second training set may include instances in which none of the alternative suggestions have been accepted.

The one or more processors 106 may further, upon receiving an input from the device 108 regarding a diagnosis, prognosis, or treatment, perform functions to improve the diagnosis or prognosis, and/or implement a treatment. For example, the one or more processors 106 may electronically record or fill out a prescription 110 and incorporate the prescription 110 into the records 104 of the patient 105, and to the database 102. In other examples, the one or more processors 106 may further communicate with an instrument by sending a signal to the instrument to further carry out a diagnosis, prognosis, or treatment. The instrument may be an imaging device such as a magnetic resonance imaging (MRI) machine 112 or a surgical machine 114. The one or more processors 106 may further transmit information about a protocol or program to be implemented at the instrument. The protocol or program may incorporate the input received from the device 108 at which suggestions were accepted or adopted. For example, if the input received from the device 108 was to add a MRI scan to a current plan, the one or more processors 106 may transmit to the MRI machine 112 a signal to active the MRI machine 112 and a program or protocol to be implemented. In such a manner, the one or more processors 106 can activate machines to further carry out a diagnosis, prognosis, or treatment. Further details of the operations of the one or more processors 106 are described below.

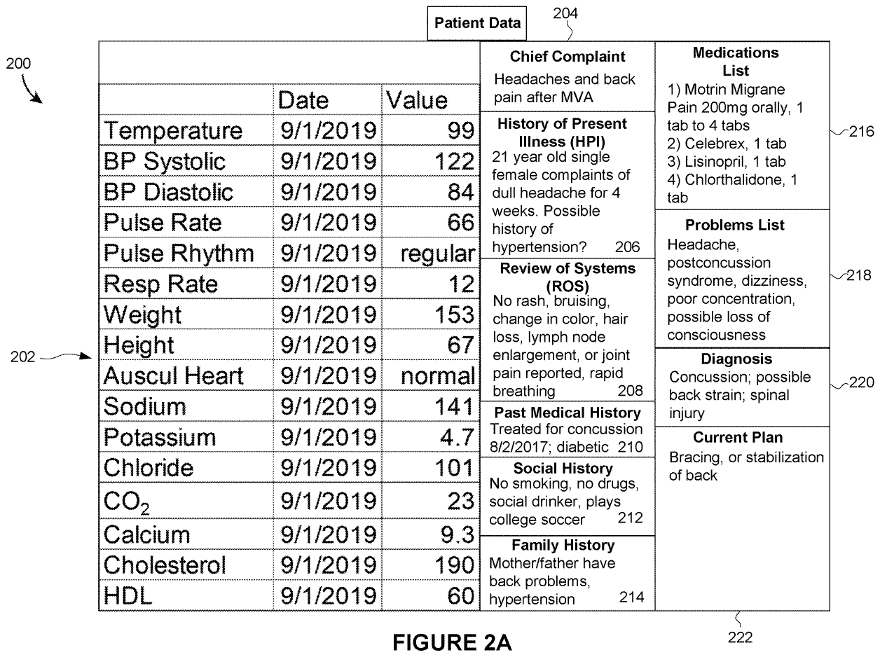

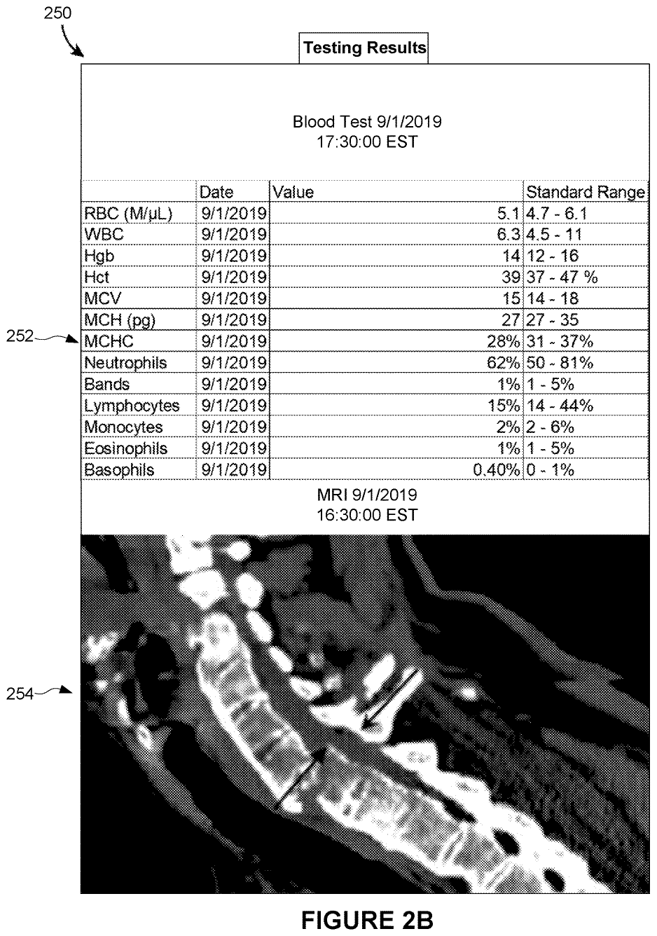

FIG. 2A illustrates an exemplary implementation of a digital health data platform, such as an EHR platform, depicting a panel 200 that includes a patient chart of the patient 105. The panel 200 may be implemented, in some embodiments, as the records 104 of the patient 105. In other words, the records 104 may be organized in a format of the panel 200. In FIG. 2A, the digital health data platform may include a clinical data window or section (hereinafter “window”) 202, a chief complaint window 204, a history of present illness (HPI) window 206, a review of systems (ROS) window 208, a past medical history window 210, a social history window 212, a family history window 214, a medications list window 216, a problems list window 218, a diagnosis window 220, and a current plan window 222. The one or more processors 106 may determine and present suggestions in any of the aforementioned windows based on any of the records 104 of the patient 105, and/or information from any of the patients 103 in the database 102. The one or more processors 106 may accept an input of numbers and/or strings into any of the aforementioned windows either manually or a selection of numbers and/or strings via a drop-down list or a drop-down menu.

FIG. 2B illustrates an exemplary implementation of a digital health data platform, such as an EHR platform, depicting a panel 250 that includes testing results of the patient 105, which may include blood test data 252 and MRI data 254. In some examples, the MRI data 254 may have been obtained in response to, or after, the one or more processors 106 presented a suggestion to incorporate a MRI in a current plan of diagnosis, prognosis, or treatment of the patient 105. The one or more processors 106 may transmit a signal to the MRI machine 112 in order to initiate a MRI scan of a back of the patient 105. Once the MRI machine 112 receives the instructions including settings and/or protocol of the MRI scan from the one or more processors 106, the MRI machine 112 may conduct the MRI scan and transmit results of the MRI scan to the one or more processors 106. The one or more processors 106 may then insert the MRI results including images into the panel 250. Although particular examples of diseases or medical conditions are mentioned for the sake of illustration, the implementation of FIGS. 2A and 2B may apply to any disease or medical condition.

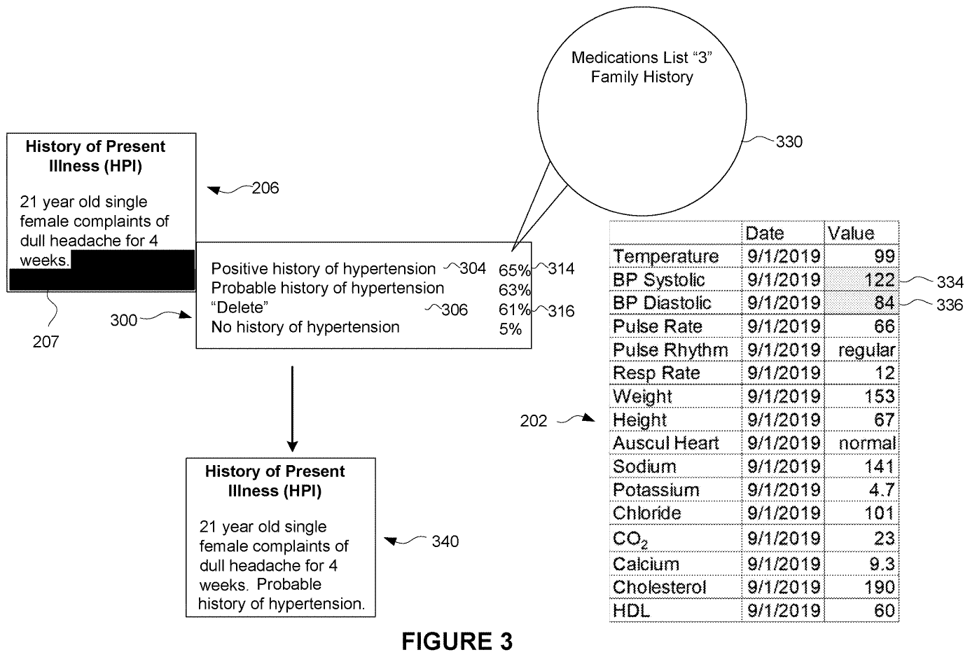

FIG. 3 illustrates an exemplary implementation of a mechanism of updating the digital health data platform by the one or more processors 106, showing how the records 104 of the patient 105 are updated following suggestions, or options, presented by the one or more processors 106. As an example, the one or more processors 106 may determine and present a suggestion to modify any of the text in the panel 200. For example, the one or more processors 106 may recognize that text somewhere in the panel 200 may be ambiguous, inaccurate, or incomplete, and present replacement text to replace or add to the ambiguous, inaccurate, or incomplete text, and/or present an option to delete the ambiguous, inaccurate, or incomplete text. In some embodiments, the one or more processors 106 may only present replacement text if a probability of reliability of any of replacement text options or suggestions satisfies a certain threshold, such as 50%.

In FIG. 3, the one or more processors 106 may determine that text in the HPI window 206 may be ambiguous and may determine replacement text based on context from the records 104 of the patient 105 and/or other data in the database 102. In particular, the one or more processors 106 may determine that text 207, “Possible history of hypertension?” from the HPI window 206 may be ambiguous. In order to determine replacement text to suggest, the one or more processors 106 may extract other data from one or both of the panels 200 and 250, and/or other sources of data of the patient 105 or in the database 102 that is potentially relevant and/or related to text 207, “Possible history of hypertension?” For example, the extracted data may include data from the medications list window 216. Lisinopril, which is known to treat hypertension, being included in the medications list window 216 may be evidence to support that the patient 105 does indeed have a history of hypertension. In addition, the extracted data may include data from the family history window 214. Data in the family history window 214 indicates that both parents have hypertension, a further testament that the patient 105 has a history of hypertension. The extracted data may further include data from the patient chart 202, indicating that systolic and diastolic blood pressures of the patient 105 are within normal ranges. These blood pressure readings may support the possibility that the patient 105 does not have a history of hypertension.

Thus, the one or more processors 106 may have to balance potentially conflicting data from the panels 200 and 250 in order to determine replacement text to suggest and present to the device 108, in place of the text 207. Here, the one or more processors 106 may determine that the data from the medications list window 216 and the family history window 214 which suggest a history of hypertension outweigh the data from the patient chart 202, which suggests no history of hypertension.

Therefore, the one or more processors 106 may determine an option 304 to be, substituting, in place of the text 207, a replacement text option “Positive history of hypertension.” The one or more processors 106 may further determine alternative options 306 which may include replacement text options “Probable history of hypertension,” or “No history of hypertension.” The one or more processors 106 may further determine that one of the alternative options 306 is to delete the text 207, “Possible history of hypertension,” without substituting any text. The one or more processors 106 may further determine a probability of relevance of each of the replacement text or deletion options. The probability of relevance may indicate an extent to which each of the replacement text or deletion options is relevant and/or accurate. For example, a probability of relevance 314 of the option 304 may be determined to be 65%, indicating that some uncertainty exists as to whether the option 304 is relevant or accurate, in part because the patient chart 202 appears to suggest no hypertension. The alternative options 306 may have lower probabilities of relevance 316 compared to the probability of relevance 314 of the option 304.

The one or more processors 106 may present the option 304 and the alternative options 314 in a window 300 having a drop-down list or drop-down menu to enable a selection of an input from the device 108. The window 300 may provide a possibility of selecting any of the option 304 or the alternative options 314, or overriding all options. If the option 304 and the alternative options 314 have been overridden, other options include keeping the text 207 as is or manually inputting a different string to replace the text 207. Thus, the device 108 may indicate that none of the option 304 and the alternative options 314 are accepted or adopted. The window 300 may be implemented either as an overlay over existing medical data or in a sidebar to a side of the existing medical data, which may be presented in the panel 200 or 250. In FIG. 3, if the input from the device 108 indicates that the “Probable history of hypertension” option were selected, the one or more processors 106 may update, as an updated HPI window 340, the previous HPI window 206 having the text 207 replaced by, “Probable history of hypertension.”

In some embodiments, the one or more processors 106 may present sources of data from any of the panels 200 or 250 from which the option 304 or the alternative options 314 were determined, inferred, or derived, as a tooltip, pop-out menu, popup window, or a hover box. As illustrated in FIG. 3, the one or more processors 106 may present a tooltip, pop-out menu, popup window, or a hover box 330 showing that the option 304 and the alternative options 314 were obtained from data in the medications list window 216, third item (“Lisinopril”) and the family history window 214. The one or more processors 106 may additionally present an additional tooltip, pop-out menu, popup window, or a hover box showing sources of data that may introduce uncertainty of any of the option 304 and the alternative options 314 being reliable. In particular, the one or more processors 106 may highlight, as entries 334 and 336, data from the patient chart 202 that appears to lead away from a history of hypertension.

The one or more processors 106 may utilize machine learning, which may incorporate neural networks such as a convolutional neural network (CNN). In some examples, the machine learning model may be trained to detect ambiguous or erroneous entries using input training data that includes ambiguous or erroneous entries, which may conflict or not exactly be consistent with other data in a patient data platform. The machine learning model may be trained to recognize and output which particular entries are ambiguous or erroneous. Part of the training process may incorporate training data having inputs that appear primarily to support a particular clinical concept and outputs which may include any data sources that are conflicting or not exactly consistent with the clinical concept. The machine learning model may be trained to further determine possible replacement strings to replace the ambiguous or erroneous entries, using the same ambiguous or erroneous entries along with the other conflicting or inconsistent data as input and the possible replacement strings as an output. For example, the machine learning model may be trained to incorporate information from the conflicting or inconsistent data while formulating options of possible replacement strings. The inputs of training data may include medical data and the outputs may include a string that incorporates at least a portion of the medical data.

The machine learning model may further incorporate reinforcement learning to cope with the stochasticity in data entries in a patient data platform to improve the option 304 and the alternative options 314 over time. For example, the reinforcement learning may leverage a reward shaping mechanism which may further include Bayesian optimization, to modify algorithms of the one or more processors 106 over time based on input from the device 108 indicating a frequency at which the option 304 or the alternative options 314 are adopted or accepted, or overridden. In some embodiments, if the device 108 provides input that tends to override most of the presented options while changing the ambiguous or erroneous entries without incorporating any of the presented options, the one or more processors 106 may adapt by presenting options that conform more closely with a style and/or substance of information of the changed entries from the device 108. As an example, the device 108 may provide input that the text 207, “Possible history of hypertension?” should be changed to “Probable history of hypertension from medications list and family history” which does not conform to any of the option 304 or the alternative options 314. The one or more processors 106 may adapt by presenting future suggestions or options that also mention or include sources of information. The machine learning model, as will be elaborated on, may also perform segmentation such as instance and semantic segmentation, and/or be trained to perform instance and semantic segmentation.

In some examples, the one or more processors 106 may adapt by presenting or providing suggestions or options that take into account which types of options have been accepted by the device 108. For example, the one or more processors 106 may take into account that a selection from the device 108 is one of the alternative options 306, “Probable history of hypertension” rather than the option 304, “Positive history of hypertension,” suggesting that less definitive language may be preferred. Thus, the one or more processors 106 may adapt by presenting options in the future that have less definitive language. The one or more processors 106 may also detect a type of language elsewhere in the panel 200 and conform to the detected type of language. In some examples, the one or more processors 106 may adapt by suggesting or presenting options that take into account relative weights to be attributed to each of the sources of data from any of the panels 200 and 250 in particular situations or in general. In the example shown in FIG. 3, the one or more processors 106 may infer that data from the medications list window 216 and the family history window 214 are to be weighted more heavily than data from the patient chart 202 in determining a history of hypertension. If additional selections from the device 108 indicate that data from the medications list window 216 and the family history window 214 are to be weighted more heavily than the patient chart 202 in other scenarios, the one or more processors 106 may accordingly weight data from the medications list window 216 and the family history window 214 more heavily while formulating its options or suggestions.

The probabilities of relevance 314 and 316 may also be adjusted based on input received from the device 108. For example, if input from the device 108 indicates that data from the medications list window 216 and the family history window 214 are to be weighted more heavily than other data at a high frequency, the probabilities of relevance of options or suggestions associated with or including such data may be adjusted to be higher. Although particular examples of diseases or medical conditions are mentioned for the sake of illustration, the implementation of FIG. 3 may apply to any disease or medical condition.

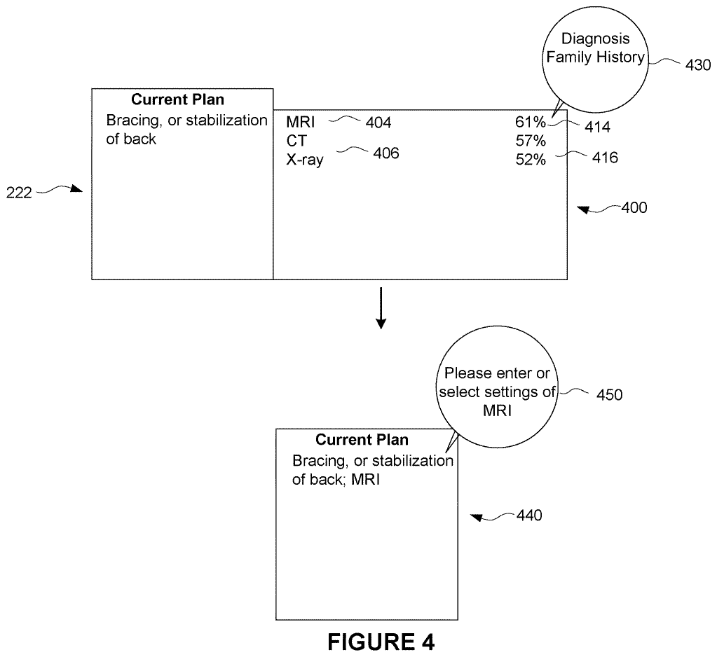

FIG. 4 illustrates an exemplary implementation of a mechanism of updating the digital health data platform by the one or more processors 106, showing how the records 104 of the patient 105 are updated following suggestions, or options, presented by the one or more processors 106. Relevant details provided with respect to other figures may also be applicable to the implementation shown in FIG. 4. In FIG. 4, the one or more processors 106 may provide options to further enrich an entry in the current plan window 222 by suggesting an option 404 of including a MRI scan, and alternative options 406 that include a CT scan or an X-ray instead. The one or more processors 106 may further present a probability of relevance 414 corresponding to the option 404 and probabilities of relevance 416 corresponding to the alternative options 406.

The one or more processors 106 may first recognize or determine that some type of imaging may be an appropriate further option to guide diagnosis, prognosis, or treatment, based on information presented in the chief complaint window 204. The one or more processors 106 may determine that no image has been recorded in the panel 250 corresponding to an applicable date such as a date indicated in the patient chart 202 of when the patient 105 was most recently seen in the clinic, and that no indication of an image has been recorded in the current plan window 222. To clarify, although the MRI data 254 is shown in the panel 250, the MRI data 254 may not have been present before the one or more processors 106 suggested an option 404 of including the MRI scan, and may only have been added afterwards. The one or more processors 106 may then determine which imaging options and/or modalities are most appropriate for a current problem, in this case, back pain.

The one or more processors 106 may present the option 404 and the alternative options 406 in a window 400 while further including a tooltip, pop-out menu, popup window, or a hover box 430 to provide a reasoning or justification for the option 404 and the alternative options 406. The window 400 may be implemented either as an overlay over existing medical data or in a sidebar to a side of the existing medical data, which may be presented in the panel 200 or 250. For example, the tooltip, pop-out menu, popup window, or the hover box 430 shows that data from the diagnosis window 220 and the family history window 214 indicates that the patient 205 has a back problem and that further diagnosis is required. The one or more processors 106 may then receive a selection from the device 108 regarding which, if any, of the option 404 or the alternative options 406 are adopted or accepted. In the example of FIG. 4, a selection from the device 108 indicates that the option 404 has been accepted. The one or more processors may then output an updated current plan window 440 that includes the option 404, “MRI.”

Subsequently, the one or more processors 106 may present, to the device 108, a prompt to select a protocol and/or settings of implementing an MRI scan. For example, the prompt may be in a form of a dialog 450. In some examples, the dialog 450 may be a pop-up window which enables a selection from predetermined settings or a manual entry from the device 108 of the protocol and/or settings to be followed. In some examples, the predetermined settings may include commonly used settings, such as settings commonly used in a diagnosis or prognosis of the current problem. In some examples, the dialog 450 may provide an option on the device 108 to select a commonly used setting while modifying specific parameters or properties of that setting. The one or more processors 106 may then communicate with an MRI machine (e.g., the MRI machine 112) and transmit the settings and/or protocol to be applied, to initialize the MRI machine 112.

The one or more processors 106 may be trained using training data having inputs of particular medical scenarios and outputs of what diagnostic or prognostic mechanisms are used in those particular medical scenarios. For example, the one or more processors 106 may be trained to recognize that MRI imaging is most commonly used in diagnosing back problems. In some examples, the one or more processors 106 may be trained to associate a reference to a back problem, for example, located somewhere in the panel 200 or 250, with an imaging modality. If the one or more processors 106 do not detect that any imaging modality is present in the panel 200 or 250, then the one or more processors 106 may determine that an option of an imaging modality should be, or is to be, presented at the device 108. However, if the one or more processors 106 do detect that an imaging modality is referred to on the applicable date somewhere in the panel 200 or 250, the one or more processors 106 may refrain from presenting an additional imaging modality, even if the imaging modality referred to is not the most commonly used one. Thus, the one or more processors 106 take into account that a practitioner is in a best position to determine certain diagnostic or prognostic options and do not interfere with the practitioner's practice.

In some examples, the one or more processors 106 may further adapt by modifying the options or suggestions presented based on feedback from the device 108 regarding which diagnostic or prognostic mechanisms are selected in certain situations. For example, if CT is most commonly selected in the situation of back problems, the one or more processors 106 may adapt by presenting CT instead of MRI as the option 404 (a primary option) rather than as one of the alternative options 406.

The probabilities of relevance 414 and 416 may also be adjusted based on input received from the device 108. For example, if the input from the device 108 indicates that CT is much more frequently used compared to MRI, the probabilities of relevance of options or suggestions associated with or including CT may be adjusted to be higher. Although particular examples of diseases or medical conditions are mentioned for the sake of illustration, the implementation of FIG. 4 may apply to any disease or medical condition.

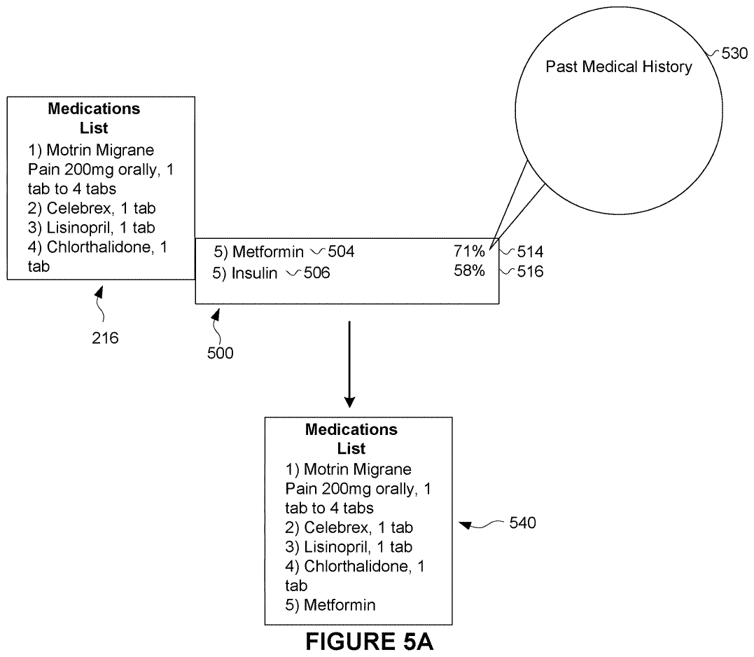

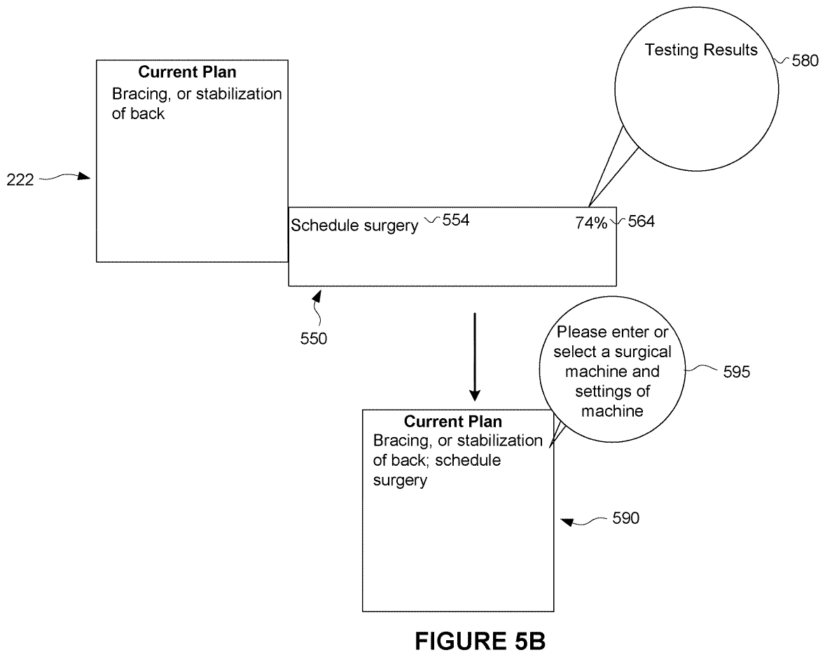

FIG. 5A illustrates an exemplary implementation of a mechanism of updating the digital health data platform by the one or more processors 106, showing how the records 104 of the patient 105 are updated following suggestions, or options, presented by the one or more processors 106. Relevant details provided with respect to other figures may also be applicable to the implementation shown in FIG. 5A. Sometimes, if the medications list window 216 does not include all medications that the patient 105 is currently being administered or prescribed, the one or more processors 106 may infer or determine other medications that the patient 105 may actually be administered or prescribed based on context presented from data in either or both of the panels 200 and 250 and/or from other data such as that included in the database 102 and pharmacy claims data.

For example, the one or more processors 106 may extract data from the past medical history window 210 that the patient 205 is diabetic, and associate a reference related to diabetes with a drug specifically tailored to diabetic patients. That is, if the one or more processors 106 fail to detect any reference to such a drug in the medications list window 216, the one or more processors 106 may determine or infer that the medications list window 216 is missing that drug. The one or more processors 106 may determine one or more particular drugs that are commonly used on diabetic patients, and/or commonly used on diabetic patients having same or similar demographic characteristics as the patient 105 and output these particular drugs as options or suggestions. Thus, the one or more processors 106 may output in a window 500 to the device 108, an option 504 which includes the drug metformin, and an alternative option 506 which includes the drug insulin, while indicating that probabilities of relevance 514 and 516 are 71% and 58%, respectively. The window 500 may be implemented either as an overlay over existing medical data or in a sidebar to a side of the existing medical data, which may be presented in the panel 200 or 250. The one or more processors 106 may further present a tooltip, pop-out menu, popup window, or a hover box 530 to provide a reasoning or justification as support for the option 504 and the alternative option 506. For example, the tooltip, pop-out menu, popup window, or the hover box 530 shows that the medications indicated in the option 504 and the alternative option 506 may be necessary based on data from the past medical history window 210. In FIG. 5A, the one or more processors 106 may detect that an input from the device 108 indicates a selection of metformin, and present an updated medications list window 540 that includes metformin.

The one or more processors 106 may be trained using a training dataset having inputs of certain problems, conditions or diseases, and outputs of what types of medications and/or particular medications associated with those problems, conditions or diseases. For example, the one or more processors 106 may recognize and/or associate types of medications that are used for diabetes, in order to determine whether the medications list window 216 is missing a medication. The one or more processors 106 may further be trained to determine which types of medications are frequently utilized to treat particular conditions or problems for particular demographics of patients. For example, the one or more processors 106 may be trained to recognize that metformin is most frequently used to treat diabetes patients of a particular demographic type same or similar to that of the patient 205 so that the one or more processors 106 will present metformin as the option 504. However, if the one or more processors 106 detect that a diabetes medication is referred to in the medications list window 216, the one or more processors 106 may refrain from presenting an option of another medication, even if that diabetes medication referred to is not metformin or is not commonly used. Thus, the one or more processors 106 take into account that a practitioner is in a best position to determine certain medications and do not interfere with the practitioner's practice.

In some examples, the one or more processors 106 may further adapt by modifying the options or suggestions presented based on feedback from the device 108 regarding which medications are selected in certain situations. For example, if metformin is most commonly selected to treat diabetic patients of a certain demographic and insulin is most commonly selected to treat diabetic patients of a different demographic, the one or more processors 106 may adapt by presenting the medications most commonly used as the option 504 depending on which demographic a patient fits in.

The probabilities of relevance 514 and 516 may also be adjusted based on input received from the device 108. For example, the higher a frequency at which a particular medication is selected to treat or address a given disease or medical condition and/or a patient demographic, the higher the probability of relevance of an option or suggestion associated with that particular medication. Although particular examples of diseases or medical conditions are mentioned for the sake of illustration, the implementation of FIG. 5A may apply to any disease or medical condition.

FIG. 5B illustrates an exemplary implementation of a mechanism of updating the digital health data platform by the one or more processors 106, showing how the records 104 of the patient 105 are updated following suggestions, or options, presented by the one or more processors 106. Relevant details provided with respect to other figures may also be applicable to the implementation shown in FIG. 5B. The one or more processors 106 may determine further options or suggestions to supplement a current entry in the current plan window 222. In some embodiments, the one or more processors 106 may determine, from other data presented in the panel 200 and/or the panel 250, that further treatment such as surgery may be required.

In some examples, the one or more processors 106 may analyze updated data in the testing results, such as the MRI data 254, to determine a presence of a particular medical condition such as a fracture. The process of determining a presence of a condition, such as a fracture, may involve semantic segmentation and/or instance segmentation to determine boundaries between different types of tissues such as bone, joints, muscle, and fat, and determine different boundaries between a same type of tissue. The one or more processors 106 may be trained using a training dataset that includes inputs of reference image data that shows boundaries among tissues of a normal patient and of a patient that exhibits a particular medical condition, and outputs indicating whether or not that reference image data is of a normal patient. For example, if a determined boundary in a current patient such as the patient 105 is not present in the reference image data of a normal patient, the one or more processors 106 may infer or determine that the determined boundary in the current patient corresponds to a fracture or other medical condition.

The performing of semantic and/or instance segmentation may be by the same machine learning model that was trained based on text inputs. In particular, the same machine learning model that was trained to detect ambiguous or erroneous entries using input training data may also be trained, or otherwise configured, to perform semantic segmentation and/or instance segmentation. In some embodiments, the machine learning model may include an ensemble machine learning model that is trained to perform both segmentation and detection of ambiguous or erroneous text entries.

If the one or more processors 106 determine the presence of a fracture or other medical condition, the one or more processors 106 may determine that further treatment beyond that provided in the current plan window 222 should be suggested. For example, the one or more processors 106 may associate a fracture with a surgical procedure. Thus, the one or more processors 106 may determine if any reference to a surgery or particular surgical procedure is included in either the panel 200 or the panel 250. If the one or more processors 106 determine that no such reference is included, the one or more processors 106 may present or suggest, in a window 550, that an option 554 of scheduling a surgery be added to the current plan window 222. The window 550 may be implemented either as an overlay over existing medical data or in a sidebar to a side of the existing medical data, which may be presented in the panel 200 or 250. A probability of relevance 564 of 74% may be presented in the window 550 and be indicative of a relative frequency of a fracture in that region of a body requiring a surgical procedure for a particular demographic. The one or more processors 106 may further present a tooltip, pop-out menu, popup window, or a hover box 580 to provide a reasoning or justification as support for the option 554. For example, the tooltip, pop-out menu, popup window, or the hover box 580 shows that the measures indicated in the option 554 may be necessary based on data from the testing results in the panel 250. In FIG. 5B, the one or more processors 106 may detect that an input from the device 108 indicates that the option 554 has been accepted. The one or more processors 106 may present an updated current plan window 590 that includes scheduling surgery.

Subsequently, the one or more processors 106 may present, to the device 108, a prompt to select a machine to conduct a surgery and protocol and/or settings of implementing a surgical procedure on the selected machine. For example, the prompt may be in a form of a dialog 595. In some examples, the dialog 595 may be a pop-up window which enables a selection from predetermined settings or a manual entry from the device 108 of the protocol and/or settings to be followed. In some examples, the predetermined settings may include commonly used settings, such as settings commonly used in a particular type of surgical procedure. In some examples, the dialog 595 may provide an option on the device 108 to select a commonly used setting while modifying specific parameters or properties of that setting. The one or more processors 106 may then communicate with the selected surgical machine (e.g., the surgical machine 114) and transmit the settings and/or protocol to be applied, to initialize the surgical machine 114.

The one or more processors 106 may be trained using training data having inputs of particular medical conditions and outputs of what measures, such as surgery or particular types of surgery, are implemented when those particular medical conditions occur. For example, the one or more processors 106 may be trained to recognize that surgery is commonly used in situations of fractures. In some examples, the one or more processors 106 may be trained to associate a fracture, as indicated or detected from somewhere in the panel 200 or 250, with a surgery. If the one or more processors 106 do not detect that any reference to a surgery or particular type of surgery is present in the panel 200 or 250, then the one or more processors 106 may determine that an option of a surgery should be, or is to be, presented at the device 108. However, if the one or more processors 106 do detect that a surgery or particular type of surgery is referred to on the applicable date somewhere in the panel 200 or 250, the one or more processors 106 may refrain from presenting an option of a surgery, even if that surgery referred to is not a most commonly used type of surgery in that scenario. In such a manner, the one or more processors 106 take into account that a practitioner is in a best position to determine certain surgical options and do not interfere with the practitioner's practice.

In some examples, the one or more processors 106 may further adapt by modifying the options or suggestions presented based on feedback from the device 108 regarding which surgical procedures are selected in certain situations. For example, if lumbar fusion is most commonly selected in the situation of a spinal or back fracture, the one or more processors 106 may adapt by presenting lumbar fusion as the option 554 in relevant scenarios. The probability of relevance 564 may also be adjusted based on input received from the device 108. For example, if the input from the device 108 indicates that lumbar fusion is most commonly used in spinal fractures, the probabilities of relevance of options or suggestions associated with or including lumbar fusion associated with spinal fractures may be adjusted to be higher. Although particular examples of diseases or medical conditions are mentioned for the sake of illustration, the implementation of FIG. 5B may apply to any disease or medical condition.

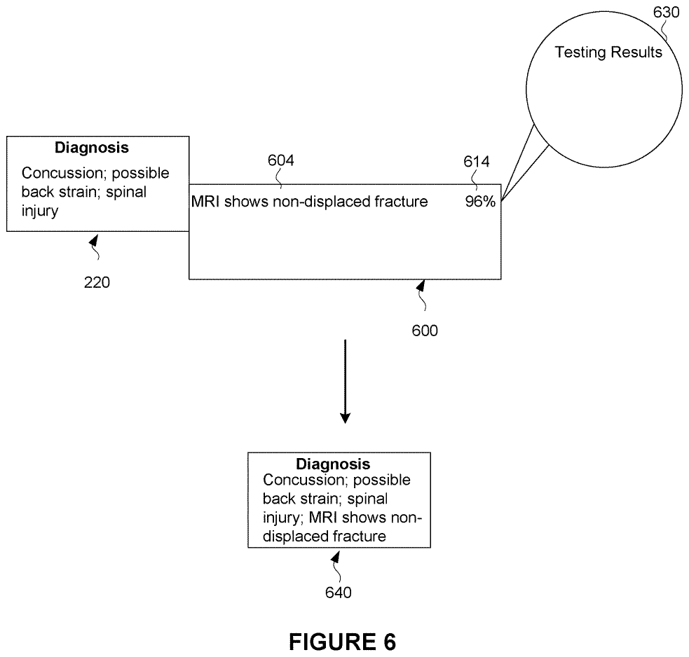

FIG. 6 illustrates an exemplary implementation of a mechanism of updating the digital health data platform by the one or more processors 106, showing how the records 104 of the patient 105 are updated following suggestions, or options, presented by the one or more processors 106. Relevant details provided with respect to other figures may also be applicable to the implementation shown in FIG. 6. The one or more processors 106 may determine further options or suggestions to supplement a current entry in the diagnosis window 220 based on updated testing data. In some embodiments, once the one or more processors 106 detect that testing data, for example, shown in the MRI data 254, shows a non-displaced fracture, the one or more processors 106 may present, to the device 108, an option or suggestion 604, in a window 600, to incorporate this information. A probability of relevance 614 of 96% may also be presented in the window 600 and indicative of a degree of certainty that the MRI data 254 and/or other data does indicate such a fracture. The window 600 may be implemented either as an overlay over existing medical data or in a sidebar to a side of the existing medical data, which may be presented in the panel 200 or 250. In FIG. 6, the one or more processors 106 may detect that an input from the device 108 indicates that the option 604 has been accepted. The one or more processors 106 may present an updated diagnosis window 640 that includes information of the MRI data.

In some examples, the one or more processors 106 may further adapt by modifying the options or suggestions presented based on feedback from the device 108. For example, if the device 108 overrides the option 604 and instead provides manually entered text, “MRI shows fracture,” which may suggest that more succinct and general language is preferred, the one or more processors 106 may adjust by providing more succinct and general options or suggestions in the future. Although particular examples of diseases or medical conditions are mentioned for the sake of illustration, the implementation of FIG. 6 may apply to any disease or medical condition.

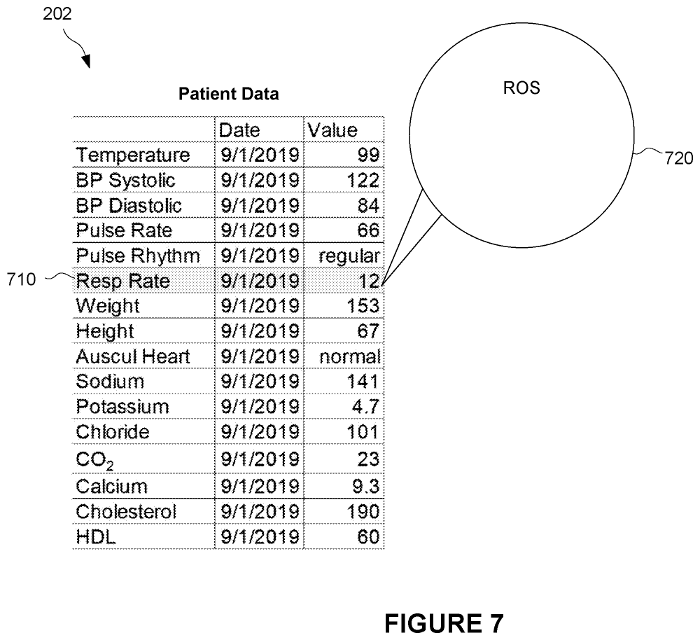

FIG. 7 illustrates an exemplary implementation of a mechanism of updating the digital health data platform by the one or more processors 106, showing how potentially erroneous entries are detected. Relevant details provided with respect to other figures may also be applicable to the implementation shown in FIG. 7. As an example, some entries in the patient chart 202, such as respiratory rate, may be commonly filled in without actual measurement by a practitioner. In other words, the practitioner may simply fill in “12,” “14,” or “16” in an effort to save time, or fill in a rough estimate without a precise measurement. Thus, the one or more processors 106 may recognize which entries are commonly filled in without actual measurement, and flag or otherwise indicate such entries. In some examples, the one or more processors 106 may flag such entries, or any entries in the patient chart 202 or elsewhere in the panel 200, if they appear to be inconsistent with other entries in the panel 200. In FIG. 7, the one or more processors 106 may flag an entry 710 indicating that a respiratory rate was 12 beats per minute because information in the ROS window 208 indicates that the patient 105 had rapid breathing, which appears to be inconsistent. The one or more processors 106 may further indicate, in a tooltip, pop-out menu, popup window, or a hover box 720, that the ROS window 208 includes potentially conflicting or contradictory data. The one or more processors 106, upon detecting potentially conflicting or contradictory data, may flag an entry that is more or most likely to be erroneous. Here, the one or more processors 106 may determine that the entry 710 in the patient chart 202 is more likely to be erroneous compared to the entry in the ROS window 208, in part because the respiratory rate is commonly filled out without measuring and the entry in the ROS window 208 requires a manual input by a practitioner.

In some embodiments, the one or more processors 106 may detect whether an entry is actually linked to or verified by a recorded sensor measurement. For example, the one or more processors 106 may determine whether a source, such as a sensor, exists to verify each entry in the patient chart 202, in other words, whether each entry can be traced to or is verified by a recorded sensor measurement. In such examples, a measurement from a sensor may be recorded and automatically populated into the patient chart 202. If the one or more processors 106 determine that an entry is not verified by a sensor measurement, the one or more processors 106 may flag that entry and/or more stringently verify, compared to other entries, whether that entry is consistent with other data, for example, in the panel 200.

The one or more processors 106 may be trained using training data having inputs of conflicting or contradictory data and outputs indicating particular data entries are erroneous and which data entries are correct. The one or more processors 106 may be trained to recognize particular factors or criteria that increase a likelihood of an entry being erroneous, such as, ease of inputting the entry, whether the entry matches a commonly inputted round number such as “12,” “14,” or “16,” and/or whether the parameter being measured in the entry is one that would otherwise be time-intensive to actually measure or is known as a parameter that is commonly inputted without an actual measurement. Although particular examples of diseases or medical conditions are mentioned for the sake of illustration, the implementation of FIG. 7 may apply to any disease or medical condition.

In some embodiments, the one or more processors 106 may be configured to identifying an entry, for example, in the patient chart 202 in which a numeral is missing a subsequent unit of measurement and which may be confusing. The one or more processors 106 may determine a particular unit of measurement to append to an end of the numeral based on the context of the entry and other data in the panels 200 and 250. The one or more processors 106 may flag the numeral that is missing a subsequent unit of measurement. However, certain numerals do not require a subsequent unit of measurement because they may be widely understood. In such scenarios, the one or more processors 106 may not flag such a numeral.

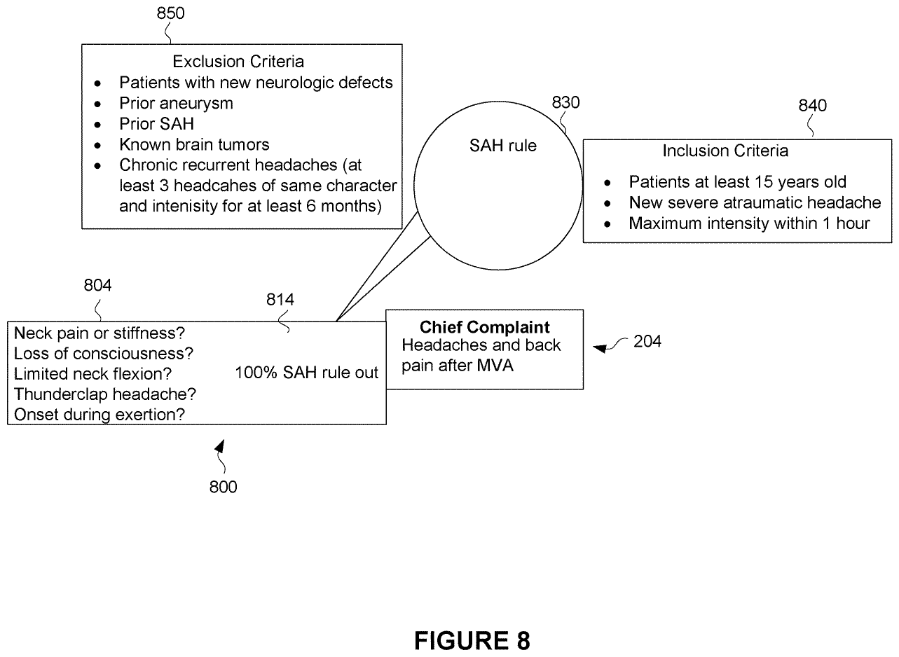

FIG. 8 illustrates an exemplary implementation of a mechanism of updating the digital health data platform by the one or more processors 106, showing a mechanism by which additional information about the patient 105 may be requested in order to determine or rule out an existence of additional conditions. Relevant details provided with respect to other figures may also be applicable to the implementation shown in FIG. 8.

As an example, the one or more processors 106 may determine that symptoms listed in the chief complaints window 204 include headaches. The one or more processors 106 may recognize that headaches may indicate other conditions such as subarachnoid hemorrhage (SAH) based on diagnostic tests, methods, or protocols programmed into the one or more processors 106. For example, the one or more processors 106 may present, to the device 108, a window 800 that includes additional questions 804 to provide further clarity to determine or rule out an existence of additional conditions. In the window 800, the one or more processors 106 may further indicate a particular condition 814 that could be confirmed or ruled out by the additional questions 804. In some examples, if the particular condition 814 cannot be confirmed or ruled out with certainty, the window 800 also indicates a likelihood that the particular condition can be confirmed or ruled out. The window 800 may be implemented either as an overlay over existing medical data or in a sidebar to a side of the existing medical data, which may be presented in the panel 200 or 250.

The one or more processors 106 may further present a tooltip, pop-out menu, popup window, or a hover box 830 to indicate a particular diagnostic test, method, or protocol used to determine whether the particular condition 814 can be confirmed or ruled out. Furthermore, the one or more processors 106 may present, as additional tooltips, pop-out menus, popup windows, or hover boxes 840 and 850, inclusion criteria and exclusion criteria of the diagnostic test, respectively. Moreover, the one or more processors 106 may present perils and pitfalls of the particular diagnostic test, method, or protocol, manners or situations in which the particular diagnostic test, method, or protocol may best be used. In some examples, the one or more processors 106 may further be programmed to execute prognostic methods, algorithms, and/or determinations of treatment options. In some examples, the one or more processors 106 may suggest which of possible prognostic methods, algorithms, and/or determinations of treatment options are most applicable to a current condition being treated. The suggestions may be presented at a sidebar to a side of the existing medical data, which may be presented in the panel 200 or 250. For each of the suggestions, the one or more processors 106 may present guidelines, whether that suggestion is still applicable or relevant in view of other diagnostic methods or tests, whether that suggestion has already been implemented, for example, if a diagnostic method has already been performed on that patient. Such an implementation takes into account that the diagnostic tests, methods, or protocols are not black and white but are blunt tools generally based on population studies and that the practitioner is in a best position to determine whether to use and a manner in which to use a particular diagnostic test, method, or protocol, and execute prognostic methods, algorithms, and/or determinations of treatment options.

Once the one or more processors 106 receives an input from the device 108, the one or more processors 106 may carry out the diagnostic test, method, or protocol and output or populate results of the diagnostic test, method, or protocol to one or more appropriate windows of the panel 200 and/or the panel 250. In some examples, a frequency or a time of populating the results to an appropriate window in one or both of the panels 200 and/or 250 may be predetermined or manually determined. In some examples, the results including a most recent result and all results in a recent timeframe, such as in the previous 24 or 72 hours, may be populated, along with native macros or dot phrases, into an appropriate window in one or both of the panels 200 and/or 250 such as, for example, the patient chart 202. Although particular examples of diseases or medical conditions are mentioned for the sake of illustration, the implementation of FIG. 8 may apply to any disease or medical condition.

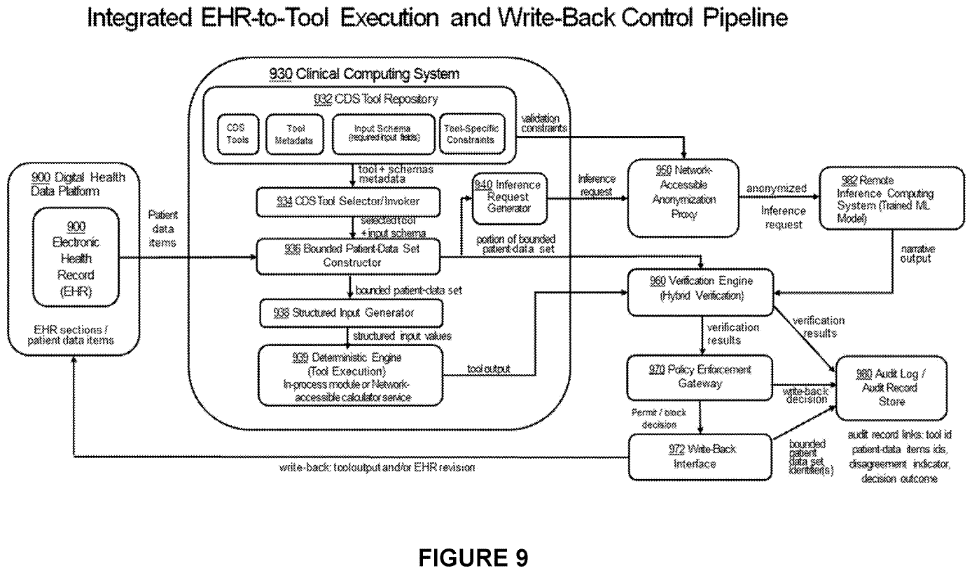

FIG. 9 illustrates an integrated EHR-to-tool execution and write-back control pipeline in which a clinical computing system 930 cooperates with a digital health data platform 900 that stores an electronic health record (EHR) for a patient. The digital health data platform 900 may be implemented as an EHR system, a digital health record repository, or a set of interoperable clinical data services that expose patient data items (e.g., vitals, labs, medication lists, problem lists, encounter notes, imaging results, and/or device-measured signals) through one or more access interfaces (for example, an API, a database connector, or an interoperability endpoint). The clinical computing system 930 accesses patient data items from the EHR and uses those patient data items as the source material for both a deterministic clinical decision-support (CDS) tool execution path and a separate machine-learning inference path, while gating any write-back to the digital health data platform 900 based on verification results.

In some embodiments, the clinical computing system 930 includes, or is coupled to, a CDS tool repository 932 that stores a plurality of CDS tools and supporting data used to invoke and validate the tools. In the illustrated example, the repository 932 includes CDS tools, tool metadata, an input schema defining required input fields, and tool-specific constraints usable for validation. The CDS tool selector/invoker 934 selects a CDS tool from the repository 932 for execution and obtains corresponding tool artifacts, including the input schema and any relevant metadata. In some embodiments, the “input schema” expresses the required input fields, expected data types, acceptable value formats, and/or unit requirements for the selected CDS tool, and may be represented as a structured specification (e.g., a JSON schema, a typed field list, a form definition, or another machine-readable schema representation) that the clinical computing system 930 can use to drive downstream selection and normalization of EHR-derived inputs.

Based on the selected tool and its input schema, a bounded patient-data set constructor 936 forms a bounded patient-data set by selecting from the EHR a subset of patient data items relevant to the required input fields. The bounded patient-data set constructor 936 may, for example, restrict retrieval and processing to only those EHR sections, codes, measurements, or note fragments that correspond to required input fields of the selected tool, thereby limiting downstream processing to a tool-scoped subset of patient information rather than operating over the entire patient chart. In some embodiments, the bounded patient-data set constructor 936 performs field-directed retrieval by issuing schema-driven queries against the digital health data platform 900, such that the set of retrieved patient data items is bounded by the required input fields and related supporting context (for example, retrieving a most recent value, a set of values in a time window, or multiple candidate values from different EHR sources for later resolution).

In some embodiments, the clinical computing system 930 further includes a structured input generator 938 that determines, from the bounded patient-data set, structured input values for the required input fields of the input schema. The structured input generator 938 may perform one or more normalization operations that transform heterogeneous EHR representations into the tool's expected input format. For example, the structured input generator 938 may map one or more patient data items to corresponding required fields, convert data types (e.g., text to numeric, categorical strings to enumerations), and/or normalize units and representations to those accepted by the selected tool's input schema. In implementations where multiple patient data items are candidates for a given required input field (for example, multiple lab results, duplicate measurements recorded in different parts of the chart, or conflicting values collected at different times), the structured input generator 938 may generate a single structured input value for that field according to a deterministic mapping and selection procedure, while retaining identifiers of the underlying patient data items used to derive that structured input value for later traceability and audit logging.

FIG. 9 further illustrates a deterministic tool execution path in which the structured input values produced by the structured input generator 938 are provided to a deterministic engine 939 that executes the selected CDS tool to produce a tool output. The deterministic engine 939 may be implemented as at least one of (i) an in-process software module executing within the clinical computing system 930 (for example, a local library implementing a clinical decision rule or calculator), or (ii) a network-accessible calculator service that executes the CDS tool remotely and returns a deterministic result. In either case, the deterministic engine 939 is configured such that, given the same structured input values and the same tool version, the engine returns the same tool output, thereby providing a stable reference result suitable for downstream verification against other outputs and for controlled write-back into the EHR.

FIG. 9 also illustrates an inference path that is logically separate from the deterministic tool execution path. An inference request generator 940 derives an inference request from at least a portion of the bounded patient-data set and provides the inference request to a network-accessible anonymization proxy 950. The anonymization proxy 950 is positioned on the network path between the clinical computing system 930 and a remote inference computing system 982 that hosts a trained machine learning model. In operation, the anonymization proxy 950 generates an anonymized inference request by removing or replacing patient-identifying tokens and withholding at least one session identifier before transmitting the anonymized inference request to the remote inference computing system 982. The remote inference computing system 982 returns a narrative output (for example, explanatory text, a proposed clinical summary, and/or a recommendation narrative) responsive to the anonymized inference request, and the narrative output is received back by the clinical computing system 930.

FIG. 9 further depicts a verification engine 960 that performs a hybrid verification using both (i) the tool output from the deterministic engine 939 and (ii) the narrative output from the remote inference computing system 982. In the illustrated embodiment, the verification engine 960 detects disagreement between the narrative output and the tool output, and also verifies one or more validation constraints for at least one of the structured input values. The validation constraints may be obtained from the CDS tool repository 932 as tool-specific constraints (as depicted by the “validation constraints” relationship), and may include, for example, permitted ranges, permissible units, completeness requirements, and/or other tool-defined input validity rules that can be checked deterministically against the structured input values used to execute the tool. By combining (a) a cross-check between the narrative output and the deterministic tool output with (b) deterministic validation of the underlying tool inputs, the verification engine 960 can produce verification results that are suitable for enforcing data integrity and reducing the risk that unverified or inconsistent content is written back to the EHR.

Based on the verification results output by the verification engine 960, a policy enforcement gateway 970 selectively permits or blocks write-back to the digital health data platform 900. In the illustrated example, the policy enforcement gateway 970 outputs a permit/block decision and controls a write-back interface 972 that performs write-back of at least one of the tool output or a revision to the EHR. In some embodiments, the write-back interface 972 writes structured artifacts to the EHR (for example, a computed risk score, an interpretation category, a time-stamped result record, a proposed problem-list update, or another EHR update object), and the policy enforcement gateway 970 ensures that such write-back occurs only when the hybrid verification indicates that applicable validation constraints are satisfied and that the narrative output does not materially disagree with the deterministic tool output (or, in some implementations, only a permitted subset is eligible for persistence). In this manner, FIG. 9 illustrates that write-back is not an unconditional automation step, but instead is mediated by a verification-driven enforcement layer that provides a technical mechanism for controlling propagation of computed or inferred outputs into the patient's longitudinal health record.

FIG. 9 further illustrates an audit log/audit record store 980 that stores an audit record associated with each attempted or completed write-back. The audit record store 980 receives at least the write-back decision and may further receive identifying information sufficient to link the decision to the specific tool invocation and data basis. In some embodiments, the audit record includes an identifier of the selected CDS tool, identifiers of patient data items in the bounded patient-data set used to determine the structured input values, the tool output, an indication of whether disagreement was detected, and a write-back decision outcome. The audit record may thereby support traceability of how a particular EHR update was derived, including which tool was executed, which underlying patient data items were relied upon, whether a narrative/tool inconsistency was detected, and whether the system permitted or blocked write-back.

Accordingly, FIG. 9 shows the end-to-end architecture and core processing flow including EHR access, tool selection from a repository with an input schema, construction of a bounded patient-data set, generation of structured input values, deterministic tool execution, anonymization-proxy routing to a remote inference computing system hosting a trained model, hybrid verification, policy-gated write-back, and audit record storage. FIG. 9 further illustrates storage of an audit record linked to the selected tool, the bounded patient-data set (including identifiers of patient data items), the tool output, an indication of disagreement detection, and the write-back decision outcome.

Compared with conventional clinical documentation assistants that send encounter context directly to a remote model and then surface the returned narrative for manual use, the described system introduces a technical control plane that governs what data may leave the local clinical environment and what outputs may be persisted back into the electronic record. In particular, the system constructs a bounded, tool-scoped patient-data set and routes any outbound inference request through a network-accessible anonymization proxy that programmatically removes or replaces patient-identifying tokens and withholds session identifiers prior to transmission. This enforces data-minimization and de-identification at the network boundary and reduces leakage of identifying information through request text and transport/session metadata, which existing systems frequently fail to address because they treat the model call as a direct client-to-service exchange.

In addition, the system improves record integrity by treating write-back to the EHR as a privileged operation that is automatically gated by machine verification rather than user trust in narrative text. A deterministic tool-execution path generates a tool output from structured inputs, and a verification engine performs hybrid checks that include detecting disagreement between the remote narrative output and the deterministic output and validating at least one input value against operational constraints before permitting persistence. When the checks fail, a policy enforcement gateway blocks write-back and records the decision along with identifiers of the tool and the specific patient data items that supported the computation. This reduces propagation of incorrect or unverifiable content into the EHR and provides a concrete, system-level mechanism for preventing integrity violations and enabling post hoc compliance and troubleshooting—capabilities not provided by systems that merely display recommendations without enforcing persistence controls.

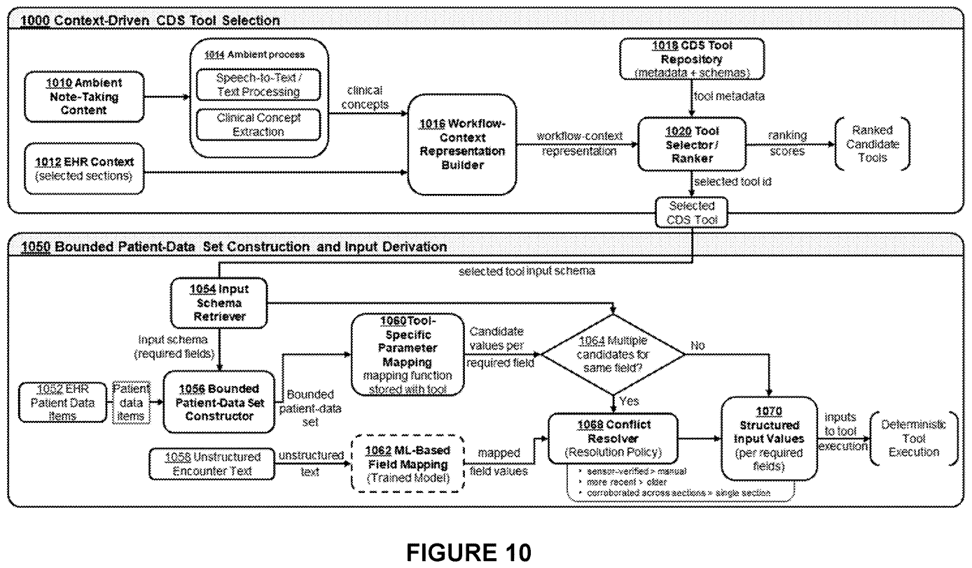

FIG. 10 illustrates an implementation of a context-driven tool-selection and input-derivation pipeline that operates on clinical encounter context to identify a clinical decision-support (CDS) tool and produce structured input values suitable for deterministic tool execution. In the illustrated example, the pipeline is organized into a first portion 1000 that selects an appropriate CDS tool based on encounter context, and a second portion 1050 that constructs a bounded patient-data set and derives structured input values corresponding to required fields of an input schema for the selected tool.

In the tool-selection portion 1000, the system receives ambient note-taking content 1010 associated with a clinical encounter and EHR context 1012 derived from selected sections of an electronic health record. The ambient note-taking content 1010 may include audio captured during a clinician-patient interaction, partial transcripts, dictation text, or other contemporaneous documentation artifacts. The EHR context 1012 may include one or more selected EHR sections, such as problem list, medications, allergies, recent vital signs, laboratory results, imaging summaries, assessment-and-plan text, or other sections that are considered relevant to tool selection. The system provides the ambient note-taking content 1010 to an ambient process 1014 that performs one or more speech-to-text or text processing operations to generate textual content, and further performs clinical concept extraction to identify one or more clinical concepts from the textual content. In some embodiments, the clinical concept extraction includes at least one of named entity recognition for problems/diagnoses/medications/labs, normalization to controlled vocabularies, detection of negation or uncertainty, and temporal or encounter-scoped attribution, thereby producing extracted clinical concepts that can be consumed by downstream selection logic.

The extracted clinical concepts output from the ambient process 1014 and the EHR context 1012 are provided to a workflow-context representation builder 1016. The workflow-context representation builder 1016 generates a workflow-context representation that encodes encounter-relevant signals in a machine-consumable form. In some embodiments, the workflow-context representation includes structured features such as coded clinical concepts, encounter type, care setting, presenting complaint, and/or one or more derived indicators from selected EHR sections; and in some embodiments the workflow-context representation further includes one or more embeddings or vector representations produced from encounter text and/or clinical concepts. The workflow-context representation is provided to a tool selector/ranker 1020, which uses tool metadata from a CDS tool repository 1018 to determine a correspondence between the workflow-context representation and candidate tools stored in the repository. The CDS tool repository 1018 stores, for each CDS tool, metadata and one or more schemas, and the tool selector/ranker 1020 uses such metadata to select a CDS tool for the encounter. In some embodiments, the tool selector/ranker 1020 produces ranked candidate tools by generating ranking scores for multiple candidate tools and selecting a top tool based on the scores, and the selected tool may be output as a selected tool identifier and/or as a selected CDS tool object for downstream processing.

In the bounded patient-data set construction and input-derivation portion 1050, the selected CDS tool from the tool-selection portion 1000 is used to drive schema-aware data selection and input derivation. The system provides a selected tool input schema to an input schema retriever 1054, which obtains an input schema that defines required fields (and, in some embodiments, optional fields) for deterministic execution of the selected tool. The input schema may define, for example, field names, expected data types, allowed units or value ranges, categorical enumerations, and/or recency or completeness requirements. The input schema retriever 1054 provides the input schema information to a bounded patient-data set constructor 1056. The bounded patient-data set constructor 1056 receives EHR patient data items 1052 and selects, from among the available EHR patient data items, those that are relevant to the required fields of the input schema, thereby constructing a bounded patient-data set. In some embodiments, the selection performed by the bounded patient-data set constructor 1056 is performed by referencing field-to-source mappings, canonical EHR locations, code-based matching (e.g., lab codes or medication identifiers), and/or section-level heuristics, such that the bounded patient-data set is tool-scoped and excludes patient data items not relevant to the required fields of the selected tool.

The bounded patient-data set is provided to a tool-specific parameter mapping component 1060, which applies a mapping function stored with, or otherwise associated with, the selected tool. The mapping function maps at least one patient data item from the bounded patient-data set to a required input field of the input schema. In some embodiments, the mapping function includes transformation logic such as unit normalization, value parsing from structured EHR fields, selection of the most recent measurement satisfying a recency constraint, resolution of categorical encodings, and/or derivation of a computed field from multiple EHR data items. The tool-specific parameter mapping component 1060 may output, for a given required input field, one or more candidate values where multiple EHR items plausibly correspond to the same field, such as multiple laboratory results, multiple vital sign entries, or conflicting values found in different sections or timestamps.

FIG. 10 further illustrates a conflict-resolution path used when multiple candidate values exist for a required field. A decision point 1064 determines whether multiple candidates exist for the same required field. If multiple candidates exist, a conflict resolver 1068 applies a resolution policy to select an input value. In the illustrated example, the resolution policy may prioritize sensor-verified measurements over manually entered values, more recent measurements over older measurements, and measurements corroborated across multiple EHR sections over measurements appearing in a single section. In some embodiments, the conflict resolver 1068 outputs not only a selected value but also provenance or selection rationale metadata, such as the source section, timestamp, verification status, or corroboration indicators. If multiple candidates do not exist at 1064, the mapping proceeds without invoking the conflict resolver. In either case, the system produces structured input values 1070 corresponding to required fields of the input schema, and the structured input values 1070 are provided as inputs to deterministic tool execution.

FIG. 10 also illustrates an optional or supplemental path for deriving one or more input values from unstructured encounter text. Unstructured encounter text 1058, which may include ambient-derived transcript text and/or clinician-entered free text, can be provided to an ML-based field mapping component 1062. The ML-based field mapping component 1062 applies a trained model to map unstructured text to one or more required input fields of the input schema, producing mapped field values that can supplement or, in some cases, provide candidate values for fields not reliably populated from structured EHR items. In some embodiments, outputs of the ML-based field mapping component 1062 are combined with outputs of tool-specific parameter mapping 1060 and, where appropriate, subjected to the same conflict-resolution logic 1068 when multiple candidates exist for a required field. In some embodiments, the ML-based field mapping component 1062 is trained using training examples that pair historical encounter text with confirmed input-field values that were used to execute a CDS tool, thereby aligning model outputs to tool-specific field semantics and reducing ambiguity in mapping.

In operation, FIG. 10 depicts a pipeline in which encounter-derived context is converted into a workflow-context representation for tool selection, and in which the selected tool's schema governs the selection of relevant patient data items and the derivation of structured inputs for deterministic execution. By separating tool selection, bounded data selection, field mapping, and conflict resolution into explicit components with defined inputs and outputs, the system can standardize how tool inputs are derived from heterogeneous EHR representations while maintaining tool-specific semantics through repository-stored schemas and mapping functions.

In some embodiments, the system improves computer operation in clinical workflows by automatically routing an encounter to an appropriate decision-support tool using machine-readable representations rather than manual navigation. For example, the system ingests heterogeneous encounter signals (e.g., ambient note-taking content and selected EHR context), applies speech-to-text or text processing, extracts clinical concepts, and generates a workflow-context representation that compactly encodes the encounter state for downstream computation. The system then performs repository-driven matching and ranking against tool metadata maintained in association with candidate tools, thereby selecting a tool identifier and corresponding tool artifacts without requiring repeated user search, scrolling, or trial execution of multiple tools. In this manner, the system reduces interactive steps and reduces computational and network overhead associated with evaluating non-relevant tools, while providing a deterministic, machine-actionable selection output that triggers a specific tool-execution path.

In some embodiments, the system further improves the reliability of automated tool execution by transforming inconsistent, duplicated, and time-variant EHR data into stable structured inputs using an enforceable conflict-resolution layer. For example, the system maps multiple candidate patient data items to a same required input field and, responsive to detecting a conflict, applies an explicit resolution policy that prioritizes objectively verifiable sources (e.g., sensor-verified measurements over manually entered values), freshness (e.g., more recent values over older values), and cross-section corroboration (e.g., values supported by multiple EHR sections over values appearing in only one section). This controlled input-derivation mechanism reduces propagation of stale or contradictory values into downstream computation, reduces rework due to later corrections, and increases repeatability of tool outputs. In some implementations, when required fields are not available as structured data, the system applies a trained machine learning model to propose candidate field values from unstructured encounter text, while still subjecting those candidates to the same structured mapping and conflict-resolution pipeline, thereby improving input coverage without turning the final result into an unconstrained free-text generation process.

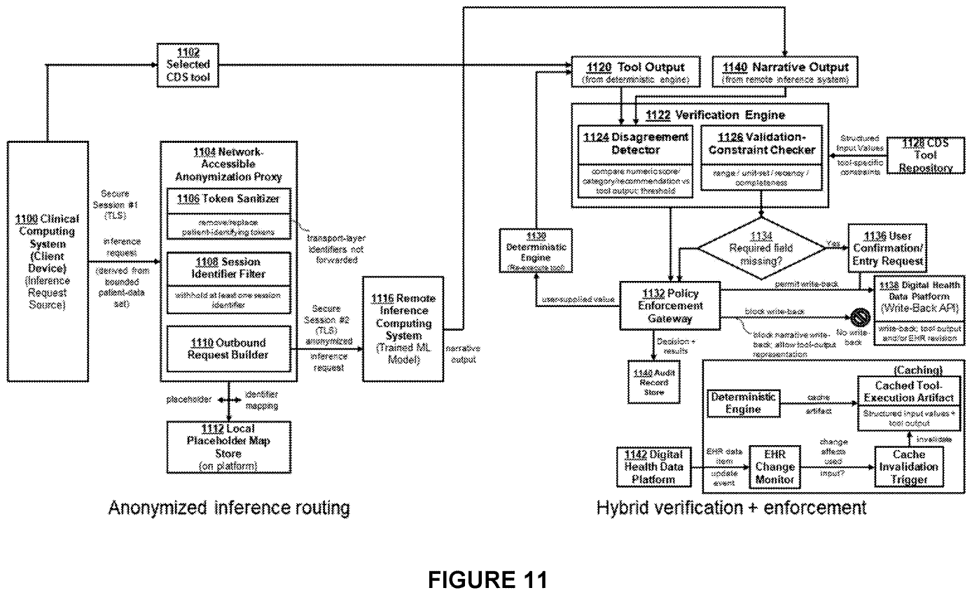

FIG. 11 illustrates a combined security and correctness enforcement architecture for integrating remote model-generated narrative content into a clinical workflow while controlling privacy exposure and preventing propagation of erroneous outputs into persistent patient records. In the illustrated embodiment, a clinical computing system interfaces with a digital health data platform that stores an electronic health record (EHR) and further interfaces with a remote inference computing system that hosts a trained machine learning model. FIG. 11 depicts a network-accessible anonymization proxy positioned logically between the clinical computing system (or a client device acting through the clinical computing system) and the remote inference computing system, such that remote inference traffic is mediated by the anonymization proxy rather than directly traversing from the clinical computing system to the remote inference computing system.

In operation, the clinical computing system forms an inference request derived from at least a portion of a bounded patient-data set, such as a structured subset of EHR data items selected for an encounter. The inference request may include contextual elements used to elicit a narrative response, such as a concise problem summary, relevant observations, medication and allergy facts, or other encounter attributes. FIG. 11 illustrates that the inference request is transmitted to the anonymization proxy, which generates an anonymized inference request before forwarding the request over a network to the remote inference computing system. In some embodiments, the anonymization proxy performs token sanitization by removing, masking, or replacing patient-identifying tokens. The token sanitization may be implemented as one or more operations including pattern-based redaction, dictionary-based detection (e.g., names, MRNs, addresses), structured-field masking for known EHR identifiers, and replacement with placeholder tokens that preserve grammatical integrity while suppressing patient identity. For example, a patient name, a medical record number, and a date of birth may be replaced with placeholders such as “[PATIENT_NAME],” “[MRN],” and “[DOB],” such that the semantic structure of the request is retained without disclosing identity.

FIG. 11 further illustrates that the anonymization proxy withholds at least one session identifier associated with communications from a client device or the clinical computing system. In some embodiments, this withholding is implemented by session termination and re-origination. For example, the anonymization proxy terminates a first secure communication session associated with the client device and originates a second secure communication session with the remote inference computing system, such that transport-layer identifiers, session tokens, or other session-correlation artifacts associated with the first secure communication session are not forwarded to the remote inference computing system. In this manner, the remote inference computing system receives an anonymized inference request that is decoupled from client-side session identity and is also sanitized to suppress patient identifiers.

In some embodiments, when placeholder tokens are used, FIG. 11 illustrates a local placeholder map store that retains a placeholder-to-identifier mapping within a trusted boundary associated with the digital health data platform and prevents transmission of the mapping to the remote inference computing system. The local placeholder map store may be implemented as a secure in-platform datastore or enclave-protected storage that is accessible to authorized components for later reconciliation within the trusted boundary, while the remote inference computing system remains unable to reverse placeholders into patient identifiers. The anonymization proxy may further enforce a one-way policy by stripping or blocking any outbound payload fields that would otherwise permit re-identification, such as patient identifiers embedded in headers, metadata, or application-level session parameters.

FIG. 11 depicts that the remote inference computing system processes the anonymized inference request using the trained machine learning model and returns a narrative output. The narrative output may include synthesized clinical text, explanatory content, or recommendations presented in natural language. Independently, FIG. 11 depicts a deterministic tool execution path that produces a tool output for the same encounter context. In some embodiments, the tool output is produced by executing a selected clinical decision-support (CDS) tool using a deterministic engine, such that the tool output includes one or more computed results that are reproducible for the same structured inputs. FIG. 11 illustrates that both the narrative output and the tool output are provided to a verification engine for hybrid verification before any write-back operation is permitted.

Within the verification engine, FIG. 11 illustrates a disagreement detector configured to detect disagreement between the narrative output and the tool output. In some embodiments, the disagreement detector parses the narrative output to identify asserted quantitative or categorical statements, such as a numeric score, a risk category, a threshold crossing, or a recommended action, and compares each parsed assertion to a corresponding value in the tool output. The disagreement detector may implement a disagreement threshold such that minor formatting differences or rounding are tolerated while material inconsistencies are flagged. For example, if the tool output yields a risk score of 3 and the narrative output asserts a risk score of 7 or asserts a “high-risk” category inconsistent with the tool output's category mapping, the disagreement detector may output a disagreement indication. In some embodiments, the disagreement detector maintains a correspondence table for associating narrative concepts to tool-output fields, such as linking “score,” “risk tier,” “meets criteria,” or “recommend admission” language to specific tool outputs.

FIG. 11 further illustrates a validation-constraint checker within the verification engine that verifies one or more validation constraints for at least one structured input value used for deterministic tool execution. In some embodiments, the validation-constraint checker applies tool-specific constraints sourced from a repository, including one or more of an allowed numeric range, an allowed unit set, a required recency threshold, and a required completeness threshold for required input fields. The validation-constraint checker may detect a unit mismatch (e.g., mmol/L vs mg/dL), a stale observation (e.g., a laboratory value outside a recency window), an out-of-range measurement likely caused by transcription error, or missing required inputs. In some embodiments, the validation-constraint checker outputs a constraint-violation indication and may attach diagnostic metadata identifying the violated constraint and the associated input value and provenance.

FIG. 11 further illustrates that the verification engine produces a verification result that is consumed by a policy enforcement gateway. In some embodiments, the policy enforcement gateway enforces write-back control to the digital health data platform based on the verification result. For example, if no disagreement is detected and the validation constraints are satisfied, the policy enforcement gateway may permit write-back of at least one of the tool output or a revision to the EHR. If disagreement is detected, FIG. 11 illustrates an enforcement action in which write-back of the narrative output is blocked while write-back of a representation of the tool output is authorized. In such embodiments, the authorized representation may omit the inconsistent portion of the narrative output and may include only deterministic, verifiable results (or a sanitized narrative fragment that is consistent with the tool output). In this manner, the write-back interface is prevented from persisting inconsistent narrative content into the EHR while still permitting storage of computed outputs that satisfy the verification criteria.

FIG. 11 optionally illustrates a missing-field handling branch in which, responsive to determining that a required input field is missing, the system generates a request for user confirmation or entry. The request may be presented through a clinical user interface to confirm, correct, or supply the missing field. After receiving confirmation or entry, the deterministic engine re-executes the selected CDS tool using the updated structured inputs, and the resulting tool output is re-submitted to the verification engine for updated hybrid verification. This feedback-controlled loop prevents tool execution from silently proceeding with incomplete or defaulted inputs and provides traceable completion behavior prior to write-back.

FIG. 11 further optionally illustrates a cached tool-execution artifact store that retains, for a selected CDS tool execution, a cache object comprising the structured input values and the corresponding tool output. The cached artifact may be used to avoid redundant deterministic executions during short-lived workflow iterations (e.g., within the same encounter session) and to stabilize downstream comparison behavior (e.g., disagreement detection) when narrative generation is repeated. FIG. 11 further illustrates an invalidation trigger responsive to detecting a change in an EHR data item that was used to determine at least one structured input value. When such a change is detected, the invalidation trigger invalidates the cached tool-execution artifact, thereby preventing stale tool outputs from being reused after underlying clinical data has changed.

In some embodiments, the architecture of FIG. 11 provides a technical improvement in privacy-preserving network communications by interposing a protocol-terminating anonymization proxy that actively removes identity-bearing tokens and suppresses session-level correlation identifiers before remote inference is invoked. In contrast to conventional systems that transmit EHR-derived prompts directly to a remote model endpoint (often coupling user sessions and patient identifiers to request metadata), the illustrated proxy decouples client sessions from remote inference sessions and confines re-identification mappings to a trusted boundary. This reduces the risk of patient re-identification by remote services and reduces the propagation of persistent identifiers across network layers, while still enabling remote inference to operate on a semantically intact request.

In some embodiments, the architecture in FIG. 11 further provides a technical improvement in data integrity and operational safety of computer-driven EHR updates by introducing a hybrid verification gate that computationally constrains what can be written back. Conventional clinical note-assist systems may treat model-generated narrative text as the primary output and may allow it to flow into the record with limited or purely manual review, which can embed inconsistent numeric scores, incorrect threshold determinations, or recommendations that do not match the underlying deterministic computation. Here, the system performs machine-checkable disagreement detection against a deterministic tool output and validates structured inputs against tool-specific constraints (e.g., range, units, recency, and completeness) before write-back is permitted. The resulting enforcement behavior blocks inconsistent narrative write-back and permits only verified content, thereby reducing the likelihood that incorrect computed facts are persisted, reducing downstream rework and correction cycles, and improving the reliability of automated EHR write-back under changing encounter data through cache invalidation tied to source-data changes.

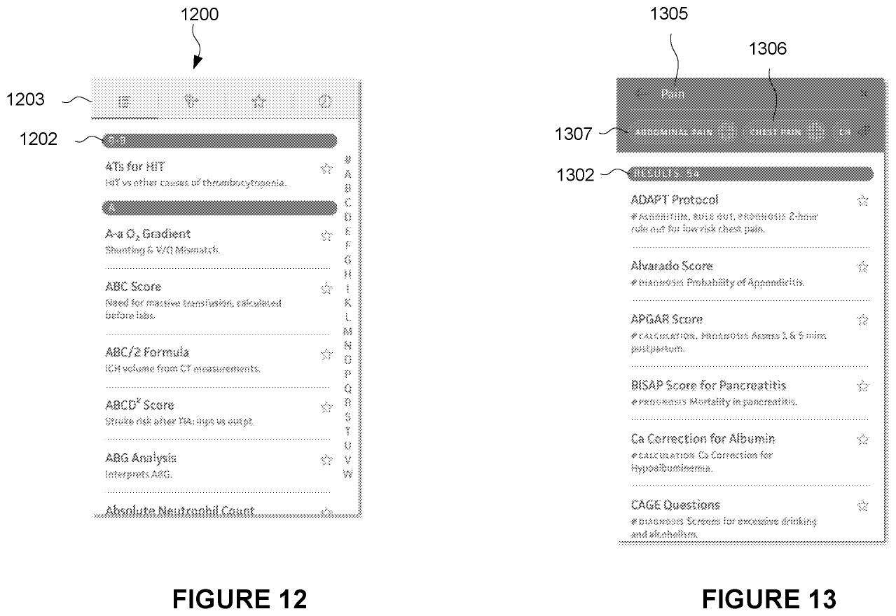

FIG. 12 illustrates a graphical interface 1200 of a diagnostic test, method, or protocol. The graphical interface 1200 may be presented on a screen of the device 108, either as an overlay over existing medical data or in a sidebar to a side of the existing medical data, which may be presented in the panel 200 or 250. The graphical interface 1200 may include a main menu 1203 and a partial listing of available diagnostic tests, methods, or protocols, prognostic methods, algorithms, and/or treatment options in panel 1202.

FIG. 13 illustrates a search interface which includes a top search box 1305, a filter/tag region 1307, and a main panel 1302 for displaying the search results. In the example of FIG. 13, if “Pain” is entered as a search query, the system conducts one or more searches in one or more databases.

The term “database” refers generically to any collection of information that is structured, either as a traditional relation table or otherwise, to enable searches and updates. A database can be located locally on a computer device from which the one or more processors 136 receive a search query and conduct a search, or remotely through a computer network. In one embodiment of the present technology, the computing device that includes the electronic screen that displays the graphical interface 1200 stores a local copy (or partial copy) of the database(s) in which the searches are conducted. The local copy enables use of the system when no internet is accessible, for instance, at an underground emergency room.

The local copy can be updated on demand or when a predetermined set of criteria are met (e.g., update frequency or schedule). In some embodiments, the program code in the system for carrying out the search and implementation of diagnostic tests, methods, or protocols, prognostic methods, algorithms, and/or determinations of treatment options can also be updated when a new version is available or when the program code changes. In one embodiment, updates to the program code and to the local copy of the database are independent of each other. That is, the system checks whether the content of the database needs updating (e.g., by comparing version number, content size, or the content itself) independently from checking whether the program code (e.g., the software package or smart phone app) needs updating. In some embodiments, each of the diagnostic tests, methods, or protocols, prognostic methods, algorithms, and/or determinations of treatment options can be independently checked and updated. For instance, each diagnostic test, method, or protocol, prognostic method, algorithm, and/or determination of a treatment option is associated with a version tag (e.g., MD5/ETag) which changes when the diagnostic test, method, or protocol, prognostic method, algorithm, and/or determination of a treatment option is updated. Thus, when the version tag for the diagnostic test, method, or protocol, prognostic method, algorithm, and/or determination of a treatment option is different between the local copy and the server, an update is appropriate and can be triggered.

One of the searches entails looking up the search query (e.g., “Pain”) against each diagnostic test, method, or protocol, prognostic method, algorithm, and/or determination of a treatment option, in the database. To this end, each entry associated with the diagnostic test, method, or protocol, prognostic method, algorithm, and/or determination of a treatment option in the database is associated with suitable annotations and categorizations, as further explained below.

As used herein, a diagnostic test, method, or protocol, prognostic method, algorithm, and/or determination of a treatment option may include a computational tool (or corresponding formula, equation or clinical decision rule) that produces a result useful in a medical setting, such as diagnosis, prognosis, health care evaluation, and treatment optimization, without limitation. The computational tool can take inputs of different types, such as numerical inputs, discrete inputs, categorical input (e.g., yes/no) or does not require an input (e.g., indication of staging of cancers).

A diagnostic test, method, or protocol, prognostic method, algorithm, and/or determination of a treatment option can be annotated with definitions or description for each of its inputs, the output, the purpose, the interpretation of the likely output, references relating to the diagnostic test, method, or protocol, prognostic method, algorithm, and/or determination of a treatment option, and originator of the diagnostic test, method, or protocol, prognostic method, algorithm, and/or determination of a treatment option. Each diagnostic test, method, or protocol, prognostic method, algorithm, and/or determination of a treatment option can also be associated with a plurality of keywords (e.g., symptoms, complaints) that are likely used for searching for the diagnostic test, method, or protocol, prognostic method, algorithm, and/or determination of a treatment option. Moreover, the keyword list can further include synonym, acronyms and abbreviations. Each of these types of information can be used for matching the diagnostic test, method, or protocol, prognostic method, algorithm, and/or determination of a treatment option with a search query.

A diagnostic test, method, or protocol, prognostic method, algorithm, and/or determination of a treatment option can be associated with one or more “tags” useful for the search. Tags are generated on different types of categorizations, such as the following:
1. Specialty (e.g. cardiology, emergency medicine)
2. Organ system (e.g. cardiovascular; renal, pulmonary)
3. Disease (e.g. heart attack, pulmonary embolism, pneumonia)
4. Chief complaint (e.g. chest pain, shortness of breath, headache)
5. Function (e.g. rule-out, diagnose, prognosticate, treat)

In one embodiment, at least a diagnostic test, method, or protocol, prognostic method, algorithm, and/or determination of a treatment option is annotated with at least one of the above categories of tags, i.e., one of specialty, organ system, disease, chief complaint, or function. In one embodiment, at least a diagnostic test, method, or protocol, prognostic method, algorithm, and/or determination of a treatment option is annotated at least with two of the above categories of tags. In some aspects, the two categories include at least function. In some aspects, the two categories include at least disease or chief complaint. In some aspects, at least a diagnostic test, method, or protocol, prognostic method, algorithm, and/or determination of a treatment option is annotated at least with three of the above categories of tags. In some aspects, the three categories include at least a function. In some aspects, the three categories include at least disease or chief complaint. In some aspects, the three categories include at least disease, chief complaint and function.

Conversely, each tag is associated with one or more diagnostic tests, methods, or protocols, prognostic methods, algorithms, and/or determinations of treatment options. Moreover, each tag can be further annotated with keywords or synonym, acronyms and abbreviations of the keywords. One way of generating such a keyword list is to incorporate the keywords from each diagnostic test, method, or protocol, prognostic method, algorithm, and/or determination of a treatment option associated with the tag.

To further facilitate search, the system can be configured to further display one or more suggested filters based on the search query, which can include the tags as well as other information. In one embodiment, when a search query is entered, the system searches through all tags and identify those that are associated with the search query (e.g., as associated keywords). The identified tags are then provided to the device 108, which may provide an input to narrow down the search results.

The filters do not have to be tags, however. In one embodiment, a filter is any category of one or more diagnostic tests, methods, or protocols, prognostic methods, algorithms, and/or determinations of treatment options. In another embodiment, the filter is a word, phrase or abbreviation that relates to a search query. The relationship can be pre-determined, such as a tag that is associated with a list of keywords. In this example, when one of the keywords is used as the search query, the tag is identified as a filter for potential use.

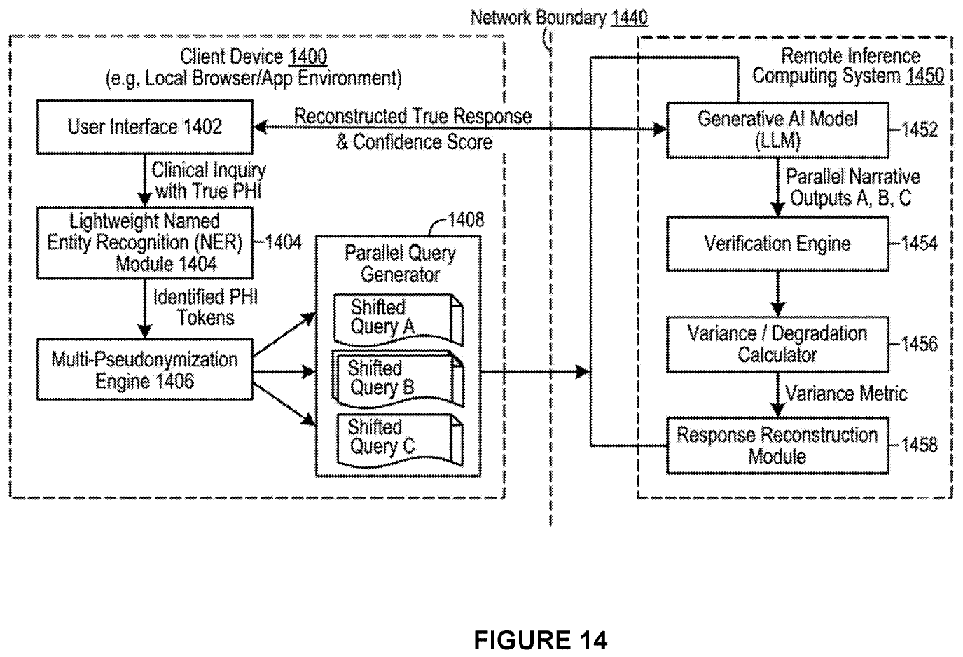

FIG. 14 illustrates a block diagram of a client-side multi-pseudonymization architecture for generating, transmitting, and evaluating parallel clinical inquiries to preserve privacy, in accordance with some embodiments. In the illustrated embodiment, a client device 1400 (e.g., a local browser environment or a mobile application environment) receives or otherwise captures a clinical inquiry that includes protected health information (PHI). The client device 1400 may be a clinician workstation, a tablet, a mobile phone, or another computing device executing a local application that enables entry of clinical text, selection of structured fields, or both. In some embodiments, the client device 1400 includes a user interface 1402 that accepts the clinical inquiry with true PHI and presents responses to a user, such as a clinician.

In operation, the clinical inquiry with true PHI is provided to a local entity-extraction process that executes within a client-side environment on the client device 1400. As illustrated, the local entity-extraction process may be implemented as a lightweight named entity recognition (NER) module 1404 that identifies PHI tokens within the clinical inquiry, such as patient names, addresses, dates of birth, phone numbers, medical record numbers, encounter identifiers, and other identifying attributes.

In some embodiments, the NER module 1404 is executed within a local script execution environment of a web browser (e.g., JavaScript or WebAssembly) or within a mobile application interface runtime, such that the clinical inquiry and identified PHI tokens remain within the client-side environment and are not transmitted over a network interface of the client device 1400.

In some embodiments, the NER module 1404 may be implemented as a microservice that operates on both unstructured free text and structured fields by applying pattern-based detection (e.g., identifier formats), dictionary-based detection, and/or model-based tagging, and outputs a set of identified PHI tokens and their locations in the clinical inquiry (e.g., character spans, token indices, or field identifiers).

Subsequently, the identified PHI tokens are provided to a multi-pseudonymization engine 1406 that generates a plurality of pseudonymized clinical inquiries from the original clinical inquiry. In some embodiments, each pseudonymized clinical inquiry replaces at least one part of the identified PHI tokens with a different set of substitute tokens, thereby forming multiple “shifted” or “fake” patient profiles that are each non-PHI while remaining medically plausible for the clinical inquiry.

For example, a patient name, address, and date of birth may be replaced with different placeholder values across multiple pseudonymized inquiries, such that one pseudonymized inquiry substitutes a first synthetic name and first synthetic address, a second pseudonymized inquiry substitutes a second synthetic name and second synthetic address, and so forth. In some embodiments, the multi-pseudonymization engine 1406 selects substitute tokens from one or more token libraries (e.g., lists of synthetic names, locations, or dates), generates substitute tokens using templated rules, or generates substitute tokens using constrained randomization. In some embodiments, the multi-pseudonymization engine 1406 enforces format consistency for each substitute token (e.g., maintaining a date format, medical record number format, or phone number format) to avoid generating malformed requests that can degrade inference quality for reasons unrelated to privacy transformation.

In some embodiments, the multi-pseudonymization engine 1406 maintains a local mapping between identified PHI tokens and substitute tokens used in each pseudonymized inquiry. For example, the mapping may store, for each PHI token, a tuple of (original token, pseudonymized token set identifier, substitute token value, token span/location). The mapping is stored locally on the client device 1400 (e.g., in protected local storage, application sandbox storage, or other client-side storage) and is not transmitted to the remote inference computing system. The mapping enables later translation of substitute tokens back into the original PHI when presenting a final clinical response within the local client environment, as described further below.

FIG. 14 further illustrates a parallel query generator 1408 that packages and dispatches the plurality of pseudonymized clinical inquiries as parallel requests, such as “Shifted Query A,” “Shifted Query B,” and “Shifted Query C.” The parallel query generator 1408 may implement one or more batching or scheduling techniques to transmit the pseudonymized clinical inquiries concurrently or near-concurrently to reduce response latency. In some embodiments, the parallel query generator 1408 assigns a local correlation identifier to each pseudonymized inquiry to enable matching returned outputs to the corresponding pseudonymized inquiry, without embedding patient identity in request metadata. In some embodiments, the parallel query generator 1408 transmits only the pseudonymized clinical inquiries across a network boundary 1440 and does not transmit the original clinical inquiry containing true PHI. In this manner, PHI remains within the client-side environment of the client device 1400 and is not transmitted to the network.

Across the network boundary 1440, FIG. 14 illustrates a remote inference computing system 1450 that hosts a trained machine learning model, such as a generative AI model 1452 (e.g., a large language model (LLM)). The remote inference computing system 1450 receives the pseudonymized clinical inquiries and produces narrative outputs respectively corresponding to the pseudonymized clinical inquiries. For example, the generative AI model 1452 may produce parallel narrative outputs A, B, and C that each include a synthesized clinical response, recommendation, and/or explanatory narrative responsive to the corresponding pseudonymized inquiry. In some embodiments, the remote inference computing system 1450 returns the narrative outputs to the client device 1400 along with correlation identifiers, timestamps, or request identifiers that allow the client device 1400 to align each narrative output with its input inquiry.

In some embodiments, the client device 1400 (or, alternatively, a verification service within the remote inference computing system 1450) evaluates a consistency among the plurality of narrative outputs to determine a reliability metric for the trained machine learning model under the applied pseudonymization. FIG. 14 illustrates a verification engine 1454 and a variance/degradation calculator 1456 that together compute a variance metric from the plurality of narrative outputs. In some embodiments, the verification engine 1454 standardizes or parses each narrative output into comparable units of analysis (e.g., extracted structured assertions, recommended actions, triage categories, medication suggestions, contraindications, or follow-up steps) and then the variance/degradation calculator 1456 computes a reliability metric based on the degree of agreement across outputs.

In some embodiments, at least two different classes of reliability metrics may be used. In a first example, the reliability metric is computed by extracting one or more clinically salient assertions from each narrative output and comparing the extracted assertions for agreement. For instance, the verification engine 1454 may extract structured fields such as “diagnosis suggestion,” “risk category,” “recommended next action,” “recommended tests,” “contraindications,” or “disposition,” and the variance/degradation calculator 1456 may compute a disagreement score based on whether these fields match across outputs A, B, and C. The disagreement score may be computed as a fraction of mismatched fields, a weighted sum of mismatches (e.g., weighting disposition recommendations more heavily than stylistic differences), or a contradiction count based on detected logical conflicts (e.g., one output recommends discharge while another recommends admission for the same presented risk facts).

In a second example, the reliability metric is computed using similarity measures over normalized text or embeddings. For instance, the verification engine 1454 may normalize each narrative output by removing PHI placeholders, standardizing units and numeric expressions, and stripping boilerplate, then compute a text similarity or semantic similarity score (e.g., cosine similarity of embedding vectors) between the outputs. The variance/degradation calculator 1456 may compute a variance measure based on dispersion of the similarity scores (e.g., low dispersion indicates high consistency, high dispersion indicates instability attributable to pseudonymization shifts). In some embodiments, the reliability metric includes both a structured-assertion agreement score and a semantic similarity score, and the client device 1400 determines that the outputs are reliable only when both scores satisfy corresponding thresholds.

In some embodiments, the client device 1400 compares the reliability metric to a reliability threshold to decide whether to generate a final clinical response and whether downstream persistence actions are permitted. In a “low variance” scenario, where the reliability metric indicates that the plurality of narrative outputs are consistent despite the different substitute token sets, the client device 1400 generates a final clinical response based on at least one of the narrative outputs. In some embodiments, generating the final clinical response includes reconstructing the final clinical response by synthesizing medical recommendations from the plurality of narrative outputs. For example, the response reconstruction module 1458 may merge common recommended actions that appear across a majority of outputs, select a canonical phrasing from the most consistent output, or compute a consensus recommendation using voting or weighted aggregation among extracted structured assertions. In some embodiments, the final clinical response is displayed on the client device 1400 alongside a confidence indicator derived from the reliability metric, such as a numerical confidence score, a categorical confidence label, or a visual indicator that the response passed the consistency evaluation.

In some embodiments, prior to displaying the final clinical response on the client device 1400, the client device 1400 translates substitute tokens present in the final clinical response back into the original PHI using the locally stored mapping. For example, if the final response includes placeholder tokens corresponding to a pseudonymized patient name or date of birth, the client device 1400 may replace those placeholders with the true patient name or date of birth from the local mapping before displaying the final response within the user interface 1402. Because the mapping is stored locally and not transmitted, re-identification occurs only within the trusted client-side environment.

In some embodiments, when the reliability metric fails to satisfy the reliability threshold (e.g., a “high variance” scenario), the client device 1400 blocks downstream use of the narrative outputs for persistence. For example, the client device 1400 may block write-back of the plurality of narrative outputs to an electronic health record and generate an alert indicating that anonymization degraded inference quality of the trained machine learning model. The alert may be presented via the user interface 1402 and may include diagnostic information such as an indication that outputs were inconsistent, a summary of key disagreements, and/or a recommendation to collect additional inputs or to execute an alternative workflow. In some embodiments, the client device 1400 may additionally prevent generation of a final clinical response when the reliability threshold is not satisfied, or may present only the deterministic tool output (when available) as a safer alternative, while withholding narrative content from persistence.

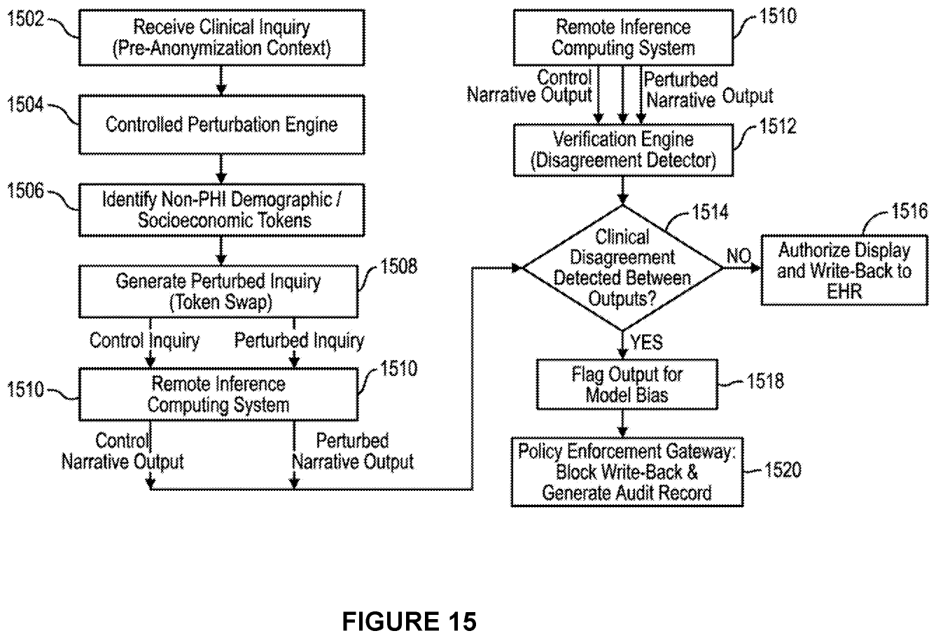

FIG. 15 illustrates a flow diagram of a controlled perturbation and bias auditing workflow for detecting algorithmic bias through clinical disagreement, in accordance with some embodiments. The workflow of FIG. 15 may be performed by a client device, a clinical computing system, a verification service, or any combination thereof, and may be used in conjunction with the multi-pseudonymization and consistency-evaluation mechanisms described elsewhere herein. In some embodiments, the workflow of FIG. 15 provides a controlled mechanism for testing whether a trained machine learning model's narrative outputs materially change when one or more demographic or socioeconomic attributes are perturbed, thereby enabling detection of bias signals that may not be apparent from a single inference.

In the illustrated embodiment, at operation 1502, a system receives a clinical inquiry including pre-anonymization context. The clinical inquiry may include unstructured encounter text and/or structured fields, and may include both clinical facts and contextual descriptors. In some embodiments, the clinical inquiry corresponds to the same inquiry used to generate the pseudonymized inquiries described with respect to FIG. 14, and is treated as a baseline inquiry for controlled perturbation.

At operation 1504, the system executes a controlled perturbation engine. The controlled perturbation engine is configured to generate one or more perturbed variants of the clinical inquiry while maintaining the clinical substance of the inquiry. In some embodiments, the controlled perturbation engine applies perturbations that are constrained to non-clinical and/or non-PHI attribute tokens, such that clinical measurements, symptoms, diagnoses, laboratory values, medications, imaging findings, and other medically salient facts remain unchanged across variants. This constraint allows the system to attribute output changes to the perturbation rather than to unrelated clinical differences.

At operation 1506, the system identifies, within the clinical inquiry, at least one token associated with a demographic or socioeconomic attribute. For example, the attribute of interest may be determined by using a locally deployed Large Language Model (LLM). In some embodiments, the demographic or socioeconomic attribute token includes a reference to race, ethnicity, gender, age group, language preference, insurance type, neighborhood or region classification, educational attainment, employment status, housing status, or other descriptors that are used in clinical documentation and that may correlate with socioeconomic context. The identification may be performed using rule-based token detection, dictionary-based matching, structured-field detection (e.g., known demographic fields), or model-based extraction. In some embodiments, the identified token is explicitly designated as non-PHI (e.g., an attribute descriptor that is not a unique identifier) and is selected such that replacement does not introduce patient-identifying information.

At operation 1508, the system generates at least one perturbed clinical inquiry by replacing the identified token with an alternative demographic or socioeconomic token. In some embodiments, the controlled perturbation engine performs a token swap that replaces a first demographic or socioeconomic descriptor with a second descriptor while preserving grammatical structure and surrounding clinical text. For example, a token indicating a first insurance type may be replaced with a different insurance type token, or a token indicating a first socioeconomic descriptor may be replaced with an alternative descriptor, while all clinical facts remain unchanged. In some embodiments, the system generates both (i) a control inquiry and (ii) a perturbed inquiry, where the control inquiry represents the baseline tokenization state and the perturbed inquiry represents a modified attribute tokenization state. In some embodiments, multiple perturbed inquiries are generated to test multiple alternative tokens or multiple attribute dimensions.

At operation 1510, the system transmits the control inquiry and the perturbed inquiry to a remote inference computing system that hosts a trained machine learning model. The remote inference computing system generates a control narrative output in response to the control inquiry and generates a perturbed narrative output in response to the perturbed inquiry. In some embodiments, the remote inference computing system is the same remote inference computing system used for generating the plurality of narrative outputs from multiple pseudonymized inquiries, and the control narrative output is generated under the same model version and inference parameters as the outputs used for consistency evaluation. In some embodiments, the control inquiry and perturbed inquiry are submitted sequentially or in parallel, and each is tagged with a local correlation identifier to enable matching of outputs to inputs without embedding patient identifiers.

At operation 1512, the system provides the control narrative output and the perturbed narrative output to a verification engine that performs disagreement detection. In some embodiments, the verification engine parses each narrative output to extract one or more clinically significant assertions or recommendations, such as disposition recommendations, urgency levels, recommended tests, contraindications, medication suggestions, or other clinical actions. The verification engine compares the extracted assertions between the control narrative output and the perturbed narrative output to determine whether the model's recommendations materially change as a function of the demographic or socioeconomic token swap.

At decision operation 1514, the system determines whether clinical disagreement is detected between outputs. In some embodiments, “clinical disagreement” refers to a material difference in a recommended action or recommendation category that would plausibly alter patient care, rather than stylistic differences in wording. For example, a clinical disagreement may include an output recommending admission versus discharge, recommending a high-risk workup versus a low-risk workup, recommending different medication classes, or recommending different urgency levels, when the underlying clinical facts are unchanged. In some embodiments, the system applies a disagreement threshold, such as a rule-based threshold (e.g., different category labels) or a score-based threshold (e.g., semantic distance exceeding a threshold for sentences tagged as “recommendations”), to avoid false positives attributable to minor phrasing differences.

If no clinical disagreement is detected at 1514, the workflow proceeds to operation 1516, where the system authorizes display and, when applicable, persistence of content. In some embodiments, authorizing persistence includes allowing write-back to an electronic health record (EHR) of a final clinical response derived from the multi-pseudonymization workflow, allowing write-back of the tool output from a deterministic engine, or both, subject to any other verification gates described herein. In some embodiments, the absence of clinical disagreement in the controlled perturbation test is treated as an additional reliability signal indicating that the model's recommendations are not sensitive to the tested attribute token.

If clinical disagreement is detected at 1514, the workflow proceeds to operation 1518, where the system flags the trained machine learning model for bias. In some embodiments, flagging includes recording an event that identifies the attribute token that was perturbed, the control and perturbed outputs, and the detected disagreement classification, and may include incrementing a bias score associated with the model version or inference endpoint. In some embodiments, the system associates the bias flag with an audit record and/or an operational monitoring dashboard to support model governance.

Following operation 1518, the workflow proceeds to operation 1520, where a policy enforcement gateway blocks write-back and generates an audit record. In some embodiments, blocking write-back includes blocking write-back of the final clinical response to an electronic health record, thereby preventing persistence of potentially biased narrative content into the patient record. In some embodiments, blocking write-back may further include blocking write-back of the control and perturbed narrative outputs and/or blocking write-back of any reconstruction output derived from the outputs under evaluation. The audit record may store, for example, the identity of the model endpoint or model version, identifiers of the control inquiry and perturbed inquiry, the replaced token and replacement token(s), a disagreement indicator, and an enforcement decision, thereby enabling traceability and compliance review.

In some embodiments, the controlled perturbation and bias auditing workflow of FIG. 15 is executed in addition to, or as a conditional branch from, the multi-pseudonymization workflow. For example, the system may first generate multiple pseudonymized inquiries and corresponding narrative outputs, evaluate consistency among those outputs, and then execute the controlled perturbation workflow to test sensitivity to a selected demographic or socioeconomic attribute. In such embodiments, comparing the control narrative output to the plurality of narrative outputs may be performed by (i) comparing the control narrative output against a consensus output derived from the plurality of narrative outputs, (ii) comparing the control narrative output against each narrative output in the plurality, or (iii) comparing the control narrative output against an aggregate representation of the plurality (e.g., a majority-vote structured assertion set), and treating a detected disagreement as a bias signal.

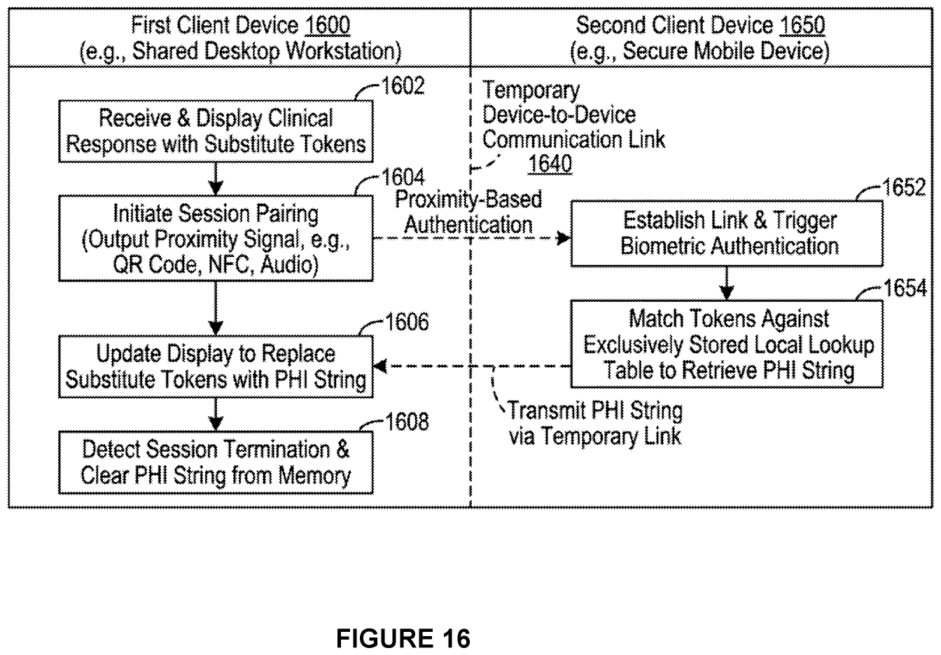

FIG. 16 illustrates a block/flow diagram of a secure, cross-device translation workflow in which a first client device 1600 (e.g., a shared desktop workstation at hospital or clinic, used by doctor, nurse, patient, or another authorized person) displays a clinical response (e.g., the response received from the remote LLM service in response to an inquiry) comprising substitute tokens and, during a paired session with a second client device 1650 (e.g., a secure mobile device), temporarily replaces the substitute tokens with corresponding PHI strings without persisting PHI on the first client device, in accordance with some embodiments. In the illustrated embodiment, the second client device 1650 maintains a secure mapping that is not available to the first client device 1600, such that substitute tokens remain non-PHI outside the second client device 1650 unless and until a proximity-based session pairing is established.

In some embodiments, the first client device 1600 receives a clinical response originating from a remote inference computing system, where the clinical response includes one or more substitute tokens in place of PHI (e.g., a patient name token, an identifier token, or another PHI placeholder). At block 1602, the first client device 1600 receives and displays the clinical response containing the substitute tokens. In some embodiments, the first client device 1600 is a shared terminal located in a clinical environment, such as a nursing station workstation, a hospital desktop, or another shared computing device where PHI persistence is undesirable. In such embodiments, the first client device 1600 displays the response in a user interface that allows a clinician to view the content while the substitute tokens remain unresolved (e.g., “PATIENT_4F21” or another anonymized identifier), thereby avoiding storage or display of PHI on the shared device in the default state.

At block 1604, the first client device 1600 initiates a session pairing protocol associated with a current secure session of the first client device 1600. In some embodiments, the current secure session corresponds to an authenticated login session, a time-limited application session, a protected browser session, or another session context that can be terminated and monitored by the first client device 1600. The session pairing protocol may include generating or outputting a proximity signal usable by a nearby second client device 1650. As illustrated, the proximity signal may include an optical machine-readable code (e.g., a QR code) presented on the display of the first client device 1600, a near-field communication (NFC) signal, a short-range wireless handshake signal (e.g., Bluetooth Low Energy (BLE)), or an ultrasonic audio signal emitted by a speaker of the first client device 1600. In some embodiments, the proximity signal encodes a session identifier or session credential associated with the current secure session, optionally including a nonce, timestamp, and/or device identifier to prevent replay.

FIG. 16 illustrates that, responsive to the proximity-based authentication, a temporary device-to-device communication link 1640 is established between the first client device 1600 and the second client device 1650. In some embodiments, proximity-based authentication includes the second client device 1650 optically scanning the optical machine-readable code displayed by the first client device 1600. In other embodiments, proximity-based authentication includes exchanging session credentials via NFC, BLE, a short-range Wi-Fi handshake, or an ultrasonic audio pairing protocol, such that both devices confirm possession of matching session credentials prior to establishing the temporary device-to-device communication link 1640. In some embodiments, the temporary device-to-device communication link 1640 is a direct local link between the devices (e.g., BLE, Wi-Fi Direct, or an application-layer peer-to-peer channel) and is configured to expire automatically when the current secure session ends or after a time limit.

In some embodiments, the session pairing protocol is performed using an out-of-band authentication code generated by a third-party authentication service. For example, the first client device 1600 may display a time-limited one-time code (e.g., a TOTP, HOTP, or other short-lived pairing code) that is generated by, or validated against, an identity provider (IdP), single sign-on (SSO) service, or multi-factor authentication (MFA) service used by the medical system. The second client device 1650 may receive the one-time code (e.g., by user entry, in-app retrieval, or IdP push approval) and transmit a pairing assertion to the first client device 1600. In such embodiments, the pairing assertion authorizes establishment of the temporary device-to-device communication link 1640 for the current secure session, while the secure mapping and PHI strings remain exclusively on the second client device 1650. In some embodiments, the one-time code is generated by an authenticator application on the second client device 1650 and validated by the first client device 1600 (or by the IdP), thereby enabling pairing without requiring optical scanning or NFC. In some embodiments, session pairing is performed using an authenticated pairing broker that does not receive PHI. For example, the first client device 1600 may request a session-scoped pairing nonce from a pairing service and display a pairing identifier (e.g., a short code) corresponding to the nonce, and the second client device 1650 may authenticate to the pairing service (e.g., via an application login, a device certificate, or managed-device attestation) and approve pairing for the displayed pairing identifier. Upon approval, the pairing service may relay only session credentials (e.g., ephemeral public keys, nonces, or session tokens) to enable establishment of the temporary device-to-device communication link 1640, without relaying any PHI strings or the secure mapping. In some embodiments, the temporary device-to-device communication link 1640 is established as a direct peer-to-peer channel; in other embodiments, the link is an end-to-end encrypted relay channel between the devices using keys negotiated during the pairing, such that any intermediary cannot access the PHI strings.

On the second client device side, FIG. 16 illustrates that the second client device 1650 establishes the temporary link and triggers user authentication at block 1652. In some embodiments, the second client device 1650 is a mobile device authenticated to a specific user (e.g., a clinician) and includes a protected execution environment and secure local storage. The user authentication may include a biometric authentication request such as fingerprint authentication, facial recognition, or another biometric factor, thereby verifying that an authorized user is present before PHI is revealed on the first client device 1600. In some embodiments, the second client device 1650 additionally requires a local passcode or application-level credential as a second factor.

At block 1654, the second client device 1650 matches one or more substitute tokens against an exclusively stored local lookup table to retrieve a corresponding PHI string. In some embodiments, the secure mapping stored exclusively on the second client device 1650 includes a locally stored lookup table that associates substitute tokens with corresponding PHI strings. In some embodiments, the lookup table is generated during a prior patient registration or prior query session in which the second client device 1650 performed client-side scrubbing and tokenization of PHI. For example, when a user enters a patient name or other PHI on the second client device 1650, the second client device 1650 may generate a substitute token by applying a one-way cryptographic function to the PHI string using a secret key stored exclusively on the second client device 1650. The substitute token may be a keyed digest value (e.g., a keyed hash or HMAC) that is stable for a given PHI string under the secret key, enabling consistent substitution within the user's environment while preventing reversal by external systems that do not possess the secret key. The second client device 1650 may store, in its secure mapping, an association between the substitute token and the original PHI string, and may further store auxiliary metadata such as an encounter identifier, patient context identifier, creation time, and/or expiration time. In some embodiments, the secure mapping is stored in a secure enclave, keychain, or application sandbox storage with encryption at rest, and is not transmitted to the first client device 1600 or to any remote system.

Upon retrieving the PHI string corresponding to the substitute token(s), the second client device 1650 transmits the PHI string to the first client device 1600 via the temporary device-to-device communication link 1640. In some embodiments, the second client device 1650 transmits only the minimum PHI necessary to resolve the displayed substitute token(s), such as a patient name for display, and does not transmit other PHI fields that are not needed for the current display context. In some embodiments, the transmission is encrypted at the application layer using session keys derived during the proximity-based authentication, such that the PHI string is protected in transit over the temporary link.

At block 1606, the first client device 1600 receives the PHI string and updates the display to replace the substitute token(s) with the corresponding PHI string for the duration of the current secure session. In some embodiments, the first client device 1600 performs the replacement only within the user interface rendering layer (e.g., a transient overlay) and avoids writing the PHI string to persistent storage, logs, clipboard buffers, or system caches. For example, the user interface may render the PHI string in-memory and may disable copy/paste of the PHI string or redact the PHI string from application telemetry, thereby reducing the risk of PHI persistence on the shared workstation.

At block 1608, the first client device 1600 detects session termination and clears the PHI string from memory. In some embodiments, session termination is detected based on expiration of an authenticated login, explicit user logout, closure of the application, inactivity timeout, or loss of the temporary device-to-device communication link 1640. Responsive to detecting termination of the current secure session, the first client device 1600 terminates the temporary device-to-device communication link 1640 and clears the PHI string from volatile memory, thereby leaving no persistent PHI on the first client device 1600. In some embodiments, the first client device 1600 further triggers clearing of any UI buffers, rendering caches, or application state that may contain the PHI string, and reverts the display to show the original substitute token(s) if the clinical response remains visible after session termination.

Accordingly, FIG. 16 illustrates an implementation in which (i) a shared desktop workstation can display anonymized clinical responses using substitute tokens, (ii) a secure mobile device holding an exclusive local mapping can selectively resolve those tokens to PHI only after proximity-based pairing and optional biometric authentication, and (iii) the resolved PHI is displayed only transiently for the duration of a secure session and is cleared upon session termination. This architecture enables cross-device usability (allowing a user to reference actual patient names across multiple patients and sessions) while preventing PHI from being exposed to or stored by remote inference systems or by shared terminals that lack the client-side secret key and secure mapping.

This design is practical in hospital environments. In many hospital environments, clinicians routinely work across a mix of shared and personal devices—for example, shared desktop workstations at nurses' stations or rounding rooms that multiple users access throughout a shift, alongside clinician mobile devices that are authenticated to a user and support biometric unlock and protected local storage-and this creates a practical tension: shared workstations are convenient for charting and viewing AI-generated narrative content, but they are not an ideal place to persist or broadly expose PHI (especially given risks like residual browser/app caches, clipboard contents, screenshots, and session handoff). FIG. 16 addresses this common workflow by keeping the shared workstation “de-identified by default” (displaying substitute tokens), while allowing a user's secure mobile device to serve as a proximity-gated, session-limited re-identification key: the clinician can pair the phone to the workstation via QR/NFC/audio for a current secure session, temporarily resolve only the needed identifiers for display, and then automatically revert to tokenized display and clear PHI from memory when the session ends or the user logs out-enabling practical, repeated multi-patient use in routine hospital settings without transmitting PHI to remote inference systems and without leaving persistent PHI on shared terminals.

The memory can include, by way of example but not limitation, random access memory (RAM), such as dynamic RAM (DRAM) and static RAM (SRAM). The memory can be local, remote, or distributed. As used in this disclosure, the term “computer-readable storage medium” is intended to include only physical media, such as memory. As used in this disclosure, a computer-readable medium is intended to include all mediums that are statutory, and to specifically exclude all mediums that are non-statutory in nature to the extent that the exclusion is necessary for a claim that includes the computer-readable medium to be valid. Known statutory computer-readable mediums include hardware (e.g., registers, random access memory (RAM), non-volatile (NV) storage, to name a few), but may or may not be limited to hardware.

The bus can also couple the processor to the non-volatile storage. The non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software on the computer system. The non-volatile storage can be local, remote, or distributed. The non-volatile storage is optional because systems can be created with all applicable data available in memory.

Software is typically stored in the non-volatile storage. Indeed, for large programs, it may not even be possible to store the entire program in the memory. Nevertheless, it should be understood that for software to run, if necessary, it is moved to a computer-readable location appropriate for processing, and for illustrative purposes, that location is referred to as the memory in this disclosure. Even when software is moved to the memory for execution, the processor will typically make use of hardware registers to store values associated with the software, and local cache that, ideally, serves to speed up execution. As used in this disclosure, a software program is assumed to be stored at an applicable known or convenient location (from non-volatile storage to hardware registers) when the software program is referred to as “implemented in a computer-readable storage medium.” A processor is considered to be “configured to execute a program” when at least one value associated with the program is stored in a register readable by the processor. Thus, the processor may include software, hardware, and firmware.

In one example of operation, a computer system can be controlled by operating system software, which is a software program that includes a file management system, such as a disk operating system. One example of operating system software with associated file management system software is the family of operating systems known as Windows® from Microsoft Corporation of Redmond, Washington, and their associated file management systems. Another example of operating system software with its associated file management system software is the Linux operating system and its associated file management system. The file management system is typically stored in the non-volatile storage and causes the processor to execute the various acts required by the operating system to input and output data and to store data in the memory, including storing files on the non-volatile storage.

The bus can also couple the processor to the interface. The interface can include one or more input and/or output (I/O) devices. The I/O devices can include, by way of example but not limitation, a keyboard, a mouse or other pointing device, disk drives, printers, a scanner, and other I/O devices, including a display device. The display device can include, by way of example but not limitation, a cathode ray tube (CRT), liquid crystal display (LCD), or some other applicable known or convenient display device. The interface can include one or more of a modem or network interface. It will be appreciated that a modem or network interface can be considered to be part of the computer system. The interface can include an analog modem, Integrated Services Digital Network (ISDN) modem, cable modem, token ring interface, satellite transmission interface (e.g. “direct PC”), or other interfaces for coupling a computer system to other computer systems. Interfaces enable computer systems and other devices to be coupled together in a network.

Several components described in this disclosure, including clients, servers, and engines, can be compatible with or implemented using a cloud-based computing system. As used in this disclosure, a cloud-based computing system is a system that provides computing resources, software, and/or information to client devices by maintaining centralized services and resources that the client devices can access over a communication interface, such as a network. The cloud-based computing system can involve a subscription for services or use a utility pricing model. Users can access the protocols of the cloud-based computing system through a web browser or other container application located on their client device.

This disclosure describes techniques that those of skill in the art can implement in numerous ways. For instance, those of skill in the art can implement the techniques described in this disclosure using a process, an apparatus, a system, a composition of matter, a computer program product embodied on a computer-readable storage medium, and/or a processor, such as a processor configured to execute instructions stored on and/or provided by a memory coupled to the processor. Unless stated otherwise, a component such as a processor or a memory described as being configured to perform a task may be implemented as a general component that is configured to perform the task at a given time or a specific component that is manufactured to perform the task. As used in this disclosure, the term ‘processor’ refers to one or more devices, circuits, and/or processing cores configured to process data, such as computer program instructions.

A detailed description of one or more implementations of the invention is provided in this application along with accompanying figures that illustrate the principles of the invention. The invention is described in connection with such implementations, but the invention is not limited to any implementation. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind, however, that all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Techniques described in this disclosure relate to apparatus for performing the operations. The apparatus can be specially constructed for the required purposes, or it can comprise a general-purpose computer selectively activated or reconfigured by a computer program stored in the computer. Such a computer program may be stored in a computer-readable storage medium, such as, but is not limited to, read-only memories (ROMs), random access memories (RAMs), EPROMS, EEPROMs, magnetic or optical cards, any type of disk including floppy disks, optical disks, CD-ROMs, and magnetic-optical disks, or any type of media suitable for storing electronic instructions, and each coupled to a computer system bus.

Unless the context requires otherwise, throughout the present specification and claims, the word “comprise” and variations thereof, such as, “comprises” and “comprising” are to be construed in an open, inclusive sense, that is as “including, but not limited to.” Recitation of numeric ranges of values throughout the specification is intended to serve as a shorthand notation of referring individually to each separate value falling within the range inclusive of the values defining the range, and each separate value is incorporated in the specification as it were individually recited herein. Additionally, the singular forms “a,” “an” and “the” include plural referents unless the context clearly dictates otherwise. The phrases “at least one of,” “at least one selected from the group of,” or “at least one selected from the group consisting of,” and the like are to be interpreted in the disjunctive (e.g., not to be interpreted as at least one of A and at least one of B).

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment, but may be in some instances. Furthermore, the particular features, structures, or characteristics may be combined in any suitable manner in one or more embodiment.

A component being implemented as another component may be construed as the component being operated in a same or similar manner as the another component, and/or comprising same or similar features, characteristics, and parameters as the another component.

1. A computer-implemented system comprising one or more processors and memory storing instructions that, when executed by the one or more processors, cause the system to:
access an electronic health record (EHR) of a patient from a digital health data platform;
select a clinical decision-support (CDS) tool from a repository of CDS tools, the selected CDS tool having an input schema defining required input fields;
construct, for the selected CDS tool, a bounded patient-data set by selecting, from the EHR, patient data items relevant to the input schema;
determine, from the bounded patient-data set, structured input values for the required input fields of the input schema;
execute the selected CDS tool using a deterministic engine, implemented as at least one of an in-process software module or a network-accessible calculator service, to generate a tool output based on the structured input values;
route, via a network-accessible anonymization proxy, an inference request derived from at least a portion of the bounded patient-data set to a remote inference computing system that hosts a trained machine learning model, wherein the network-accessible anonymization proxy generates an anonymized inference request by removing or replacing patient-identifying tokens and withholding at least one session identifier before transmitting the anonymized inference request to the remote inference computing system, and receives a narrative output from the remote inference computing system;
perform, by a verification engine, a hybrid verification comprising (i) detecting disagreement between the narrative output and the tool output and (ii) verifying one or more validation constraints for at least one of the structured input values; and
based on results of the hybrid verification, selectively permit or block, via a policy enforcement gateway, write-back of at least one of the tool output or a revision to the EHR to the digital health data platform.

2. The system of claim 1, wherein selecting the CDS tool comprises:
obtaining ambient note-taking content associated with a clinical encounter;
converting the ambient note-taking content into textual content using one or more speech or text processing operations;
extracting one or more clinical concepts from the textual content;
generating a workflow-context representation based on the one or more clinical concepts and at least one portion of the EHR; and
selecting the CDS tool from the repository based on a correspondence between the workflow-context representation and tool metadata for the CDS tool.

3. The system of claim 1, wherein determining the structured input values comprises mapping at least one patient data item to a required input field using a tool-specific parameter mapping function stored in association with the selected CDS tool in the repository,
wherein in response to multiple candidate patient data items corresponding to a same required input field, the determining comprises resolving a conflict among the multiple candidate patient data items according to a resolution policy.

4. The system of claim 1, wherein the verification engine verifies the one or more validation constraints by applying a tool-specific constraint defined in the repository, the tool-specific constraint comprising:
an allowed numeric range,
an allowed unit set,
a required recency threshold, or
a required completeness threshold for the required input fields.

5. The system of claim 1, wherein detecting disagreement between the narrative output and the tool output comprises:
determining that the narrative output asserts a numeric score, category, threshold crossing, or recommended action that is inconsistent with a corresponding value in the tool output by more than a disagreement threshold.

6. The system of claim 1, wherein the anonymization proxy withholds the at least one session identifier by:
terminating a first secure communication session with a client device, and
originating a second secure communication session with the remote inference computing system, such that transport-layer identifiers of the first secure communication session are not forwarded to the remote inference computing system.

7. The system of claim 1, wherein removing or replacing patient-identifying tokens comprises:
replacing at least one patient identifier with a placeholder token,
storing a placeholder-to-identifier mapping locally to the digital health data platform, and
preventing transmission of the placeholder-to-identifier mapping to the remote inference computing system.

8. The system of claim 1, wherein the system stores, for the selected CDS tool, a cached tool-execution artifact comprising the structured input values and the tool output, and
the system invalidates the cached tool-execution artifact responsive to detecting a change to an EHR data item that was used to determine at least one of the structured input values.

9. The system of claim 1, wherein determining the structured input values comprises:
applying a trained machine learning model to map unstructured encounter text to required input fields of the input schema, and
wherein the trained machine learning model is trained using training examples that pair (i) historical encounter text and (ii) corresponding confirmed input-field values used to execute the CDS tool.

10. A computer-implemented method for privacy-preserving clinical decision support, comprising:
receiving, at a client device, a clinical inquiry comprising protected health information (PHI);
executing, within a client-side environment of the client device, a local entity-extraction process to identify the PHI within the clinical inquiry, wherein the PHI stays within the client-side environment;
generating, by the client device, a plurality of pseudonymized clinical inquiries, wherein each of the plurality of pseudonymized clinical inquiries replaces the identified PHI with a different set of substitute tokens;
transmitting, by the client device, the plurality of pseudonymized clinical inquiries to a remote inference computing system hosting a trained machine learning model;
receiving, from the remote inference computing system, a plurality of narrative outputs respectively corresponding to the plurality of pseudonymized clinical inquiries;
evaluating a consistency among the plurality of narrative outputs to determine a reliability metric for the trained machine learning model; and
responsive to determining that the reliability metric satisfies a reliability threshold, generating a final clinical response based on at least one of the plurality of narrative outputs.

11. The method of claim 10, wherein the local entity-extraction process executed within the client-side environment of the client device comprises a Named Entity Recognition (NER) model, and wherein executing the local entity-extraction process comprises:
executing the NER model within a local script execution environment of a web browser or mobile application interface on the client device, thereby preventing transmission of the PHI over a network interface of the client device.

12. The method of claim 10, wherein generating the final clinical response comprises:
reconstructing the final clinical response by synthesizing medical recommendations from the plurality of narrative outputs; and
displaying the final clinical response on the client device alongside a confidence indicator derived from the reliability metric.

13. The method of claim 10, further comprising:
identifying, within the clinical inquiry, at least one token associated with a demographic or socioeconomic attribute;
generating at least one perturbed clinical inquiry by replacing the at least one token with an alternative demographic or socioeconomic token;
transmitting the at least one perturbed clinical inquiry to the remote inference computing system to generate a perturbed narrative output; and
comparing the perturbed narrative output to the plurality of narrative outputs to detect bias.

14. The method of claim 13, further comprising:
responsive to detecting a clinical disagreement between the perturbed narrative output and the plurality of narrative outputs based on the comparing, flagging the trained machine learning model for bias; and
blocking write-back of the final clinical response to an electronic health record.

15. The method of claim 10, further comprising:
storing, locally on the client device, a mapping between the identified PHI and the substitute tokens;
prior to displaying the final clinical response on the client device, translating the substitute tokens present in the final clinical response back into the identified PHI using the locally stored mapping.

16. A computer-implemented method for secure, cross-device translation of anonymized clinical data, comprising:
receiving, at a first client device, a clinical response originating from a remote inference computing system, wherein the clinical response comprises at least one substitute token in place of protected health information (PHI);
displaying, on a display of the first client device, the clinical response containing the at least one substitute token;
initiating, by the first client device, a session-pairing protocol corresponding to a current secure session of the first client device;
establishing a temporary local communication link with a second client device in response to a proximity-based authentication between the first client device and the second client device during the session-pairing protocol;
receiving, at the first client device via the temporary device-to-device communication link, a PHI string corresponding to the at least one substitute token, wherein the second client device generates the PHI string by matching the at least one substitute token against a secure mapping stored exclusively on the second client device; and
updating the display of the first client device to replace the at least one substitute token with the PHI string for a duration of the current secure session.

17. The method of claim 16, wherein initiating the session-pairing protocol comprises:
generating, by the first client device, an optical machine-readable code corresponding to the current secure session; and
displaying the optical machine-readable code on the display of the first client device,
wherein the proximity-based authentication comprises the second client device optically scanning the optical machine-readable code.

18. The method of claim 16, further comprising:
prior to the second client device transmitting the PHI string via the temporary device-to-device communication link, triggering a biometric authentication request on the second client device to verify an identity of an authorized user.

19. The method of claim 16, further comprising:
terminating the temporary device-to-device communication link and clearing the PHI string from memory of the first client device upon detecting termination of the current secure session, thereby leaving no persistent PHI on the first client device.

20. The method of claim 16, wherein the secure mapping stored exclusively on the second client device comprises:
a locally stored lookup table associating substitute tokens with corresponding PHI strings, wherein the substitute tokens are generated by applying a one-way cryptographic function to respective PHI strings using a secret key stored exclusively on the second client device.
